(19) United States
(12) Patent Application Publication
(10) Pub. No.: US 2018/0298436 A1
(43) Pub. Date: Oct. 18, 2018

(54) Methods and Systems for Controlling DNA, RNA and Other Biological Molecules Passing Through Nanopores

(71) Applicant: UNIVERSAL SEQUENCING TECHNOLOGY CORPORATION, Stoughton, MA (US)

(72) Inventors: Ming Lei, Sharon, MA (US); Robert Roorda, Hingham, MA (US); Zhoutao Chen, Carlsbad, CA (US); Nga Ho, Newton, MA (US); Yong Wang, Sharon, MA (US)

(21) Appl. No.: 15/767,428

(22) PCT Filed: Oct. 31, 2016
(86) PCT No.: PCT/US2016/059794

§ 371 Date: Apr. 11, 2018

Related U.S. Application Data
(60) Provisional application No. 62/248,756, filed on Oct. 30, 2015.

(51) Int. Cl. C12Q 1/6869; G01N 33/487

(52) U.S. Cl. CPC C12Q 1/6869; G01N 33/48721

(57) ABSTRACT

The present disclosure provides, in one aspect, a device and a method for unit sequencing and/or analysis of a molecular sequence comprising attaching the molecular sequence to a plate and controlling the progression of the molecular sequence through a pore of a nanopore chip, wherein the separation distance between the nanopore chip and the scan plate is controlled by a precision mechanical drive, and the molecular sequence is sensed as it progresses through the nanopore.

### BACKGROUND

### Field

The present disclosure relates generally to methods of controlling the movement of molecules, such as DNA (Deoxyribonucleic Acid), RNA (Ribonucleic Acid) and proteins as well as other biological molecules passing through nanopores so that they can be detected, characterized and/or sequenced.

Since 454 Life Sciences launched the first 2^{nd} generation DNA sequencing system in 2005, DNA sequencing has become one of the hottest technologies with a rapidly growing market. The major feature of the 2^{nd} generation DNA sequencing is its massive parallel sequencing capacity with super high throughput and short read length. It is much faster and cheaper than the 1^{st} generation technology, i.e., Sanger's method. However, its short read length makes de novo assembly, whole genome phasing, and structural variant analysis very difficult, limiting its capability to resolve complex DNA regions with repetitive or heterozygous sequences. The short read length also makes computations like sequencing entire RNA transcripts or some gene sequences in metagenomics projects difficult or impossible. A significant future application of DNA sequencing is clinical diagnostics, which requires even faster, cheaper and more accurate sequencing technologies. There is tremendous interest in searching for new DNA sequencing technologies capable of long read length (>>1000 bases), fast run time (<1 hour per genome) and low cost (<$300 per genome). Nanopore based DNA sequencing technology is the most promising 3^{rd} generation technology that could satisfy these requirements.

The concept of nanopore sequencing was developed based on the work of John Kasianowicz et al using the α-hemolysin pore in 1996. The α-hemolysin pore has a 1.5 nm diameter at its narrowest point which is sufficiently large to allow a single strand DNA (ssDNA) to pass through it but too small for double strand DNA (dsDNA). If a pore is positioned in a membrane between two pools of ionic buffer and a bias voltage is applied across the membrane, then an ionic current will be detected through the pore. When an ssDNA passes through the pore, the ionic current will be partially blocked. The magnitudes of blockage is distinct for different DNA bases. By measuring the ionic current blockages as the ssDNA passes through the pore the bases can be sequenced. The same principle can be applied to determine the sequences of RNA, polypeptide (amino acid), or other linear molecules. Other nanopore DNA base sensing methods are also under development, such as recognition tunneling (Lindsay and Zhang, 2008, 2016), and nanowire FET (Han et al, 2014).

The first patent describing DNA sequencing using a nanopore was published by Akeson et al in 2001. Since then, it attracted huge interest from both academic research labs and industrial R&D. Nanopores that can be used for DNA sequencing include biological (protein) pores held on lipid bilayer membrane, such as alpha-hemolysin pores (Kasianowicz et al, 1996, Akeson et al, 2001, Stoddart et al, 2015), MspA (Mycobacterium smegmatis porin A) pores (Derrington et al 2010, Pavlenok et at, 2012), and CsgG pores (curli specific genes G) (Goyal et al, 2014), and synthetic nanopores, including solid state nanopores and graphene nanopores (Wanunu, 2012). So far, the most difficult problem for DNA sequencing using a nanopore is that the DNA molecules pass through nanopores very fast, approximately 1 μs per base, which makes reliable base sensing with single base resolution very difficult or impossible. To have a DNA successfully sequenced, the DNA translocation speed has to be slowed down at least 1000 times to about 1 ms per base or longer. The preferred speed range for reliable single base resolution with the current base sensing technologies is 3 ms to 10 ms per base.

A variety of methods for controlling or slowing down DNA translocation speed have been proposed ever since the concept of nanopore sequencing had emerged in 1990's. These methods can be divided into the following six categories: (1) biological methods, such as molecular motors (helicase, polymerase, etc.) (Akeson et al, 2011) and dsDNA hairpin or complementary probe DNA method (Sauer-Budge et al, 2003, Derrington et al, 2010, Wanunu 2012); (2) buffer conditioning methods, such as lowering the buffer temperature (Yeh et al, 2012, Fologea et al 2005), using lithium chloride salt instead of potassium chloride (Kowalczyk et al, 2012) or increasing buffer viscosity (Fologea et al, 2005); (3) chemical methods (for synthetic nanopores), such as nanopore surface charge modification or polymer binding to the nanopore (Wanunu and Meller, 2007, Keyser, 2011); (4) electronic methods, such as DNA transistor technology (Polonsky et al, 2007, 2011) or transverse electric field dragging (Tsutsui et al, 2012); (5) Mechanical methods, such as optical tweezers (Keyser et al, 2006, Keyser 2011), magnetic tweezers (Peng and Ling, 2009) and atomic force microscopy (AFM) (King and Golovchenko, 2005), and (6) Electro-mechanical, such as integrating a piezo layer in the synthetic nanopore fabrication (Peng et al, 2011). All these methods have either problems in controlling DNA movement or difficulties in fabrication and instrumentation. The molecular motor method moves DNA at the speed of specific enzyme's intrinsic processing rate which is not easily controlled. It is suitable for biological pores only and requires complicated biochemistry for the attachment of enzymes to each pore. The dsDNA hairpin/complementary probe methods slow down DNA movement sporadically without effective speed control and require tremendous efforts on sample processing. The buffer conditioning methods can slowdown DNA translocation only five to ten times, far from the required 1000 times. The chemical methods and the transverse electronic field dragging method have no mechanism to realize any accurate DNA translocation speed control. The DNA transistor technology demonstrated a mechanism on how to control the DNA base movement inside a synthetic nanopore computationally, but it is difficult to realize because it requires a very complicated nanopore structure, it is very difficult to fabricate and integrate with base sensing. The optical and magnetic tweezers as well as the AFM methods can control a single DNA molecule passing through nanopores. It is good for academic research but far from sufficient for large scale inexpensive high-throughput DNA sequencing and other commercial applications. The electro-mechanical method proposed by Peng et al (2011) uses a piezoelectric layer in synthetic nanopore fabrication to control the nanopore size inside the nanopore so that the DNA translocation can be slowed down, however, it does not provide a mechanism on how to precisely control the DNA movement.

Thus, an improved system for controlling the movement of DNA as well as other polymers through nanopores is needed.

### SUMMARY

The present disclosure is directed toward methods and systems for controlling the translocation rate of a molecule through a nanopore with sufficient precision and at an appropriate rate to enable base unit sequencing. This disclosure broadly applies to the base unit sequencing and/or molecular structure analysis of DNA, RNA, protein and other charged biological molecules or non-charged biological molecules with charged tags, both natural and synthesized. Synthetic, non-biological molecules may also be analyzed.

In one embodiment the target molecule (e.g. ssDNA fragment to be sequenced) is attached to a magnetic bead via a linker molecule of the same type of properly chosen length and composition (e.g. a single strand λ-DNA or a synthesized DNA oligo). Electric force pulls the charged target molecule through the nanopore until its progress is impeded by the magnetic bead which is too big to pass through the nanopore. By applying a magnetic field, the magnetic bead and the attached linker can be pulled away from the nanopore toward the surface of a scan plate placed near the nanopore and perpendicular to the nanopore axis. The magnetic bead is then held against the scan plate either solely by the strength of the magnetic field or by a chemical bond or by other means. The DNA and linker are stretched under the electric force acting on the DNA bases inside the nanopore. Intra-molecular tension is built up and a force equilibrium is established between the intra-molecular tension and the electric force.

By moving either the scan plate or the nanopore substrate using a precision linear stage, the target molecule can be pulled out of the nanopore or inserted into the nanopore at the speed required for base unit sequencing. The target molecule does not move freely through the nanopore but rather is held taut under the constant tension from the opposing forces as it progresses. As the target molecule passes through the nanopore its bases are sensed and recorded by an appropriate base sensing method, such as ionic current blockage method, recognition tunneling method or other methods. Part of the linker molecule may be used as a calibration template for the sequencing if desired. Multiple target molecules can be sequenced with a single nanopore or with a plurality of nanopores (nanopore arrays).

In some embodiments of this disclosure, the linker molecule may have a linker node located where the linker attaches to the target DNA. The linker node is a molecular structure that prevents entry into the nanopore and is generally larger than the entrance of the nanopore, and in certain embodiments at least twice as large as the nanopore entrance size. The linker node can be a large protein (such as an antibody, a NeutrAvidin or a streptavidin), or a large polymer complex, or even a non-magnetic bead. The linker molecule is not required to be of the same kind of molecule as the target molecule. It can be a dsDNA, a ssDNA, or a RNA, either from nature or being synthesized, or a cellulose fiber or other flexible linear polymers. When the target DNA is pulled into the nanopore under an electric field, the linker node will function as the stopper or brake for the DNA translocation. With this linker+linker node configuration, a relatively weak magnetic field can be applied initially to lift the magnetic bead attached to one end of the linker molecule while the target molecule and the linker node are still held tight at the nanopore by the electric force. By floating the magnetic bead straight above the nanopore before it contacts the scan plate, a perfect alignment is achieved for the bead, linker molecule and the target molecule, allowing for an accurate sequencing of the target molecule.

In some embodiments of this disclosure, the target molecule is attached to the scan plate directly or via a linker molecule but without a magnetic bead. By placing the scan plate less than one linker molecule length from the nanopore substrate the target molecule can be pulled into the nanopore by the electric field. When the molecule movement is impeded by the attachment to the scan plate, it can be moved out or retracted back into the nanopore at a controlled steady speed that is slow enough for accurate base unit sensing. A plurality of target molecules can be attached laterally across the scan plate. The scan plate and nanopore substrate can be displaced laterally in an area scanning pattern to engage different molecules with the nanopore and such that they can be sequenced.

In some embodiments of this disclosure, the scan plate surface can be patterned with micro-spots or patches for target molecule attachment and these micro-patterns are precisely aligned with nanopores on a nanopore array chip. Each spot corresponds to one nanopore on the nanopore chip. Each spot may contain plurality of target molecules. After each sequencing run, the already sequenced molecule is pulled out from the nanopore and the scan plate or nanopore chip moves laterally in a scan mode to align another target molecule to enter the nanopore to be sequenced. Hence, at the end, all or the majority of the target molecules can be sequenced.

In some embodiments of this disclosure, micro-pillars with either sharp end or flat end are attached or micro-fabricated onto the scan plate surface facing the nanopore substrate. The target molecules are attached to the end of these micro-pillars directly or through a linker molecule or via a magnetic bead. These pillars are aligned with nanopores on the nanopore chip. Target molecules are sequenced with the controlled movement of the scan plate or the nanopore chip plate.

In another embodiment the target molecule (e.g. an ssDNA fragment) is attached to a magnetic bead via a linker molecule of the same type (e.g. a natural or synthesized ssDNA) of properly chosen length and composition. Electric force pulls the charged target molecule through the nanopore until its progress is stopped by the magnetic bead which is too big to pass through the nanopore. By applying a magnetic field, the magnetic bead and the attached DNA molecule can be pulled away from the nanopore toward the surface of a container wall placed near the nanopore with a distance from the nanopore sufficiently larger than the linker molecule and the target molecule combined. By solely controlling the balance of forces acting on the bead and the target DNA molecule (namely the magnetic field strength and the electric force of the nanopore), the DNA molecule can be pulled out at a speed slow enough for base sensing.

It is critical to maintain a constant force balance from the beginning of the DNA movement so that the DNA can be moved at a desired, predictable speed. This can be done by completely eliminating or minimizing the sticking forces acting on the magnetic bead when it is near or in contact with the nanopore entrance. The major source of the sticking force comes from the electric charge on the bead. By making the magnetic bead non-charged, the sticking force can be greatly reduced and a relatively smooth movement of the DNA molecule through the pore can be maintained. As the bead moves, the target molecule will be stretched under force, and an intra molecular tension will be built up. A force equilibrium is established and maintained as the target molecule proceeds through the nanopore at a controlled speed. Thus the target molecule does not move freely through the nanopore but rather is held taut under the tension from the opposing forces as it progresses. As the target molecule passes through the nanopore its bases are sensed and recorded by an appropriate base sensing method. The linker molecule of the same kind may be used as the calibration of the target molecule sequencing. Another function of the linker molecule is to provide a buffer zone so that any possible jitters of the DNA movement at the start can be lessened or dampened out. A plural form of target molecules can be sequenced with a single nanopore or a plural form of nanopores.

In some embodiment of this disclosure, for the magnetic force controlled DNA movement, a linker node is introduced as the connection between the linker molecule and the target molecule. The linker node is generally larger than the nanopore entrance, in some embodiments at least twice as large as the nanopore entrance so that it will function as the stopper or brake as the DNA translocate through the nanopore under the bias voltage. Since now the linker node is at the nanopore, it is required to be non-charged while the magnetic bead is not required to be non-charged. The linker node can be a large protein, such as an antibody, NeutrAvidin, streptavidin, or any polymer complex larger than the nanopore entrance or even a non-magnetic bead.

In another embodiment of this disclosure, the target molecule may be attached to the magnetic bead or the scan plate directly without using a linker molecule either for the mechanical controlled movement or magnetic force controlled movement approaches.

An embodiment is a system for controlling movement of a charged linear molecule comprising a substrate positioned between a cis space and a trans space, a nanopore in the substrate through which at least a portion of the charged linear molecule can pass from the cis space to the trans space, a scan plate located in the cis space to which directly or indirectly a first end of the charged linear molecule is attached, an actuator for controlling the distance between the substrate and the scan plate such that they can be moved with nanometer precision, and a bias source for applying a bias voltage between the cis space and the trans space to direct a second end of the charged linear molecule to enter into the nanopore. In another embodiment the system includes an attachment system that assists the attachment of the charged linear molecule to the scan plate such that the charged linear molecule can move with the scan plate. In another embodiment, the substrate is a nanopore chip comprising a plurality of nanopores positioned in a planar arrangement wherein each nanopore is substantially equidistant from the surface of the scan plate. In another embodiment, the nanopore comprises a biological pore, or a synthetic pore, or a combination thereof. In an embodiment, the biological pore is selected from the group consisting of an alpha-hemolysin pore, a MspA pore, a CsgG pore, a modified version thereof, and a combination thereof. In an embodiment, the synthetic pore is made from silicon nitride (Si₃N₄), silicon dioxide (SiO₂), aluminum oxide (Al₂O₃), boron nitride (BN), graphene, molybdenum disulfide (MoS2) or a polymer or a hybrid thereof. In an embodiment, the attachment system comprises a chemical bond, either covalent or non-covalent, and either reversible or non-reversible. In an embodiment, the chemical bond is selected from the list comprising a biotin-streptavidin bond, an amide bond; a phosphodiester bond, ester bond, disulfide bond, imine bond, aldehyde bond, hydrogen bond, hydrophobic bonds, and a combination thereof. In an embodiment, the attachment system comprises a magnetic bead, which is attached to the first end of the charged linear molecule, and the magnetic bead is made from one of the following materials: (a) paramagnetic, (b) super-paramagnetic, (c) ferromagnetic, or (d) diamagnetic. In an embodiment, a controllable magnet comprises an electromagnet, an adjustable permanent magnet, a group of magnets, or a combination thereof and wherein the controllable magnet is configured to attract the magnetic bead towards the scan plate and to hold the magnet bead against the scan plate. In an embodiment, the attachment system comprises a flexible linker molecule, and the flexible linker molecule is attached to the first end of the charged linear molecule at one end and attached to the scan plate at other end. In an embodiment, the flexible linker molecule is selected from the group consisting of a single stranded nucleic acid, a double stranded nucleic acid, a polypeptide chain, a cellulose fiber or any flexible linear polymer, either natural, modified or synthesized, and a combination thereof. In an embodiment, the flexible linker molecule is the same kind of molecule as the charged linear molecule. In an embodiment, the attachment system further comprises a linker node which is disposed between the flexible linker molecule and the charged linear molecule; and wherein the linker node is configured to block the linker molecule from entering the nanopore. In an embodiment, the linker node is a protein selected from the group consisting of an antibody, an enzyme, a NeutrAvidin, a streptavidin, and an avidin, or a polymer complex, or a particle or a bead, or a combination thereof. In an embodiment, a flexible linker molecule is disposed between the charged linear molecule and the magnetic bead. In an embodiment, the flexible linker molecule is selected from the group consisting of a single stranded nucleic acid, a double stranded nucleic acid, a polypeptide chain, a cellulose fiber or any flexible linear polymer, either natural, modified or synthesized, and a combination thereof. In an embodiment, the flexible linker molecule is the same kind of molecule as the charged linear molecule. In an embodiment, the attachment system further comprises a linker node which is disposed between the flexible linker molecule and the charged linear molecule; and wherein the linker node is configured to block the linker molecule from entering the nanopore. In an embodiment, the linker node is a protein selected from the group consisting of an antibody, an enzyme, a NeutrAvidin, a streptavidin, an avidin, a polymer complex, a particle, a non-magnetic bead, and a combination thereof. In an embodiment, the system comprises a detector for determining the identity or characteristics of individual base units of the charged linear molecule as they pass through the nanopore, wherein the base units of the charged linear molecule can be detected by their effect on the ionic current blockage, or recognition tunneling, or field-effect transistor, or other base sensing methods, or a combination thereof. In an embodiment, the system comprises a micro-pillar attached or microfabricated onto the scan plate that has either a pointed end or a flat-bottom end, which is configured to allow the attachment of the first end of the charged linear molecule. In an embodiment, the micro-pillar is an array of micro-pillars on the scan plate, laterally positioned to match an array of nanopores on the substrate. In an embodiment, the actuator comprises a precision linear motion stage that is configured to control the distance between the scan plate and the substrate such that the charged linear molecule can be pulled out or inserted into the nanopore at a steady rate that enables accurate base unit sequencing. In an embodiment, the rate is about 0.5 ms per base unit or slower. In an embodiment, the rate is from about 3 ms to about 20 ms per base unit. In an embodiment, the precision linear motion stage comprises a linear stage driven by a piezo-electric effect drive with nanometer or sub-nanometer precision. In an embodiment, the actuator comprises a coarse precision actuator that is coupled to the scan plate or the substrate by mechanical reduction allowing for nanometer or sub-nanometer precision movement of the scan plate or the substrate. In an embodiment, the coarse precision actuator comprises a micrometer or a sub-micrometer servo motor. In an embodiment, the system further comprises an adjustment stage with micrometer precision that is coupled to the scan plate or the substrate that is configured to move the object laterally and/or vertically for pre-sequencing position adjustment. In an embodiment, a plurality of charged linear molecules is attached to the scan plate randomly. In an embodiment, a plurality of charged linear molecules is attached to a patterned area on the scan plate, wherein the plurality of charged linear molecules in the patterned area is laterally aligned with a plurality of nanopores on the substrate. In an embodiment, the system further comprises a secondary adjustable magnet that is configured to remove the magnetic bead from the scan plate. In an embodiment, the charged linear molecule is a nucleic acid sequence or a polypeptide sequence; and wherein the nucleic acid sequence is selected from the list consisting of single stranded DNA, double stranded DNA, single stranded RNA, oligonucleotide, a sequence comprising a modified nucleotide, and a combination thereof.

An embodiment is a method for controlling movement of a charged linear molecule comprising providing a scan plate and a substrate plate placed substantially parallel and aligned laterally with each other; attaching a first end of the charged, linear molecule to the scan plate, either directly or indirectly; aligning the charged linear molecule with a nanopore in the substrate plate by an adjustable mechanical apparatus; directing a second end of the charged linear molecule to the nanopore in the substrate plate by an electric force; moving the charged linear molecule through the nanopore by adjusting the distance between the scan plate and the substrate plate; maintaining an intra-molecular tension in the charged linear molecule by adjusting the electric force during the sequencing process. In an embodiment, the adjustable mechanical apparatus comprises a single axis or a multi-axis linear stage with micrometer or sub micrometer precision. In an embodiment, the adjusting the distance between the scan plate and the substrate plate comprises moving the scan plate and/or the substrate plate with an actuator. In an embodiment, the actuator comprises a linear stage driven by a piezo-electric effect drive with nanometer or sub-nanometer precision. In an embodiment, the actuator comprises a coarse precision actuator that is coupled to the scan plate or the substrate by mechanical reduction providing for nanometer or sub-nanometer precision movement of the scan plate or the substrate plate. In an embodiment, the coarse precision actuator comprises a micrometer or a sub-micrometer precision servo meter. In an embodiment, the electrical force is achieved by an electrical bias apparatus which is configured to apply a bias voltage across the substrate plate allowing a current through the nanopore and is further configured to pull the charged linear molecule through the nanopore. In an embodiment, a flexible linker molecule is disposed between the charged linear molecule and the scan plate; and wherein the flexible linker molecule is selected from the list consisting of a single stranded nucleic acid, a double stranded nucleic acid, a polypeptide chain, a cellulose fiber or any flexible linear polymer, either natural, modified or synthesized, and a combination thereof. In an embodiment, a linker node is disposed between the linker molecule and the charged linear molecule and the linker node is configured to block the linker molecule from entering the nanopore, and wherein the linker node is a protein selected from the list consisting of an antibody, an enzyme, a NeutrAvidin, a streptavidin, and an avidin, or a polymer complex or particle or bead, a portion thereof, and a combination thereof. In an embodiment, the method further comprises attaching the first end of the charged linear molecule to a magnetic bead; wherein the magnetic bead is chosen from the list consisting of a super-paramagnetic bead, a paramagnetic bead, a ferromagnetic bead, and a diamagnetic bead; aligning the charged linear molecule with the nanopore by applying a magnetic field to attract the magnetic bead towards the scan plate while the electric force maintains the engagement of the charged linear molecule with the nanopore; wherein the magnetic field is from an electromagnet or an adjustable permanent magnet, or a group of magnets, or a combination thereof; wherein the magnetic bead contacts the surface of the scan plate substantially orthogonally aligned above the nanopore and is held tightly against the scan plate by the magnetic field such that the magnetic bead and the charged linear molecule move substantially with the scan plate. In an embodiment, a flexible linker molecule is disposed between the charged linear molecule and the magnetic bead; and wherein the flexible linker molecule is selected from the list consisting of a single stranded nucleic acid, a double stranded nucleic acid, a polypeptide chain, a cellulose fiber or any flexible linear polymer, and a combination thereof, either natural, modified or synthesized. In an embodiment, a linker node is disposed between the linker molecule and the charged linear molecule and the linker node is configured to block the linker molecule from entering the nanopore; and wherein the linker node is a protein selected from the list consisting of an antibody, an enzyme, a NeutrAvidin, a streptavidin, and an avidin, or a polymer complex or a non-magnetic particle/bead, or a combination thereof; and wherein aligning the charged linear molecule with a nanopore in the substrate plate further comprises setting a distance between the scan plate and the substrate plate larger than the length of the linker molecule; applying a magnetic field such that the magnetic force on the magnetic bead is sufficient to lift the magnetic bead, but not sufficient to substantially lift the linker node; wherein the linker node is engaged at the nanopore by the electric force, reducing the distance between the scan plate and the substrate plate to be smaller than the length of the linker molecule; wherein the magnetic bead contacts the scan plate and is held by the scan plate substantially orthogonally aligned above the nanopore, resulting in a substantial orthogonal alignment between the bead, the linker molecule and the charged linear molecule. In an embodiment, the charged linear molecule is randomly distributed on the scan plate, either attached directly or indirectly. In an embodiment, a plurality of the charged linear molecules is distributed in a patterned area on the scan plate either attached directly or attached indirectly, wherein the plurality of the charged linear molecules in the patterned area on the scan plate is substantially laterally aligned with a plurality of the nanopores on the substrate plate. In an embodiment, the substrate plate is a nanopore chip comprising a plurality of nanopores positioned in a planar arrangement such that each nanopore is substantially equidistant from the surface of the scan plate. In an embodiment, the scan plate has a micro-pillar facing the nanopore, which has either a pointed end or a flat end; and wherein the first end of the charged linear molecule is attached to the micro-pillar, either directly or indirectly. In an embodiment, the scan plate has an array of micro-pillars that match an array of nanopores on the substrate plate. In an embodiment, the charged linear molecule is a nucleic acid sequence or a polypeptide sequence; and wherein the nucleic acid sequence is selected from the list consisting of single stranded DNA, double stranded DNA, single stranded RNA, oligonucleotide, a sequence comprising a modified nucleotide, and a combination thereof.

### BRIEF DESCRIPTION OF THE FIGURES

FIG. 1 illustrates a simplified implementation of the disclosure with a DNA fragment attached to a linker molecule passing through a nanopore and spanning the gap between a nanopore chip and scan plate

FIG. 2 illustrates a subassembly in which a scan plate and a nanopore chip are pressed apart by a compressed element. The subassembly is shown inside a rigid frame that includes a precision analysis actuator that can push the scan plate and nanopore chip closer together.

FIG. 3 illustrates an alternative dynamic chamber subassembly with separate springs and seals.

FIG. 4 illustrates options for rigidly connecting a scan plate and a nanopore chip together using precision stages, including a coarse adjustment stage and a separate precise analysis stage, as well as rigid frame components.

FIG. 5 shows the types of nanopores, synthetic and biological

FIG. 6 shows nanopore designs with limiting aperture and integrated base sensing electronics. The limiting aperture allows only one DNA molecule entering the pore, preventing double or multiple DNA copies enter the pore simultaneously. The limiting aperture can be a size restricting structure or be formed by either chemical restriction, or electric or magnetic restrictions or combination of them.

FIG. 7 shows different pore array or nanopore chip configurations

FIG. 8 shows DNA attachment on scan plate surface (a) random distribution and (b) patterned attachment areas

FIG. 9 illustrates DNA attachment to micro-pillars (a) a pointed micro-pillar, showing the compensation of DNA-pore misalignment using a flexible linker molecule, and (b) a flat bottomed micro-pillar, using multiple DNA attachment to minimize lateral mis-alignment.

FIG. 10 shows different cis and trans electrode and micro-pillar arrangements

FIG. 11 illustrates piecewise repeated sequencing scheme

FIG. 12 illustrates automatic DNA alignment with a magnetic bead.

FIG. 13 shows examples of DNA sample preparations for automatic alignment

FIG. 14 shows automatic DNA-nanopore alignment with linker molecule and a linker node bead

FIG. 15 shows automatic DNA-nanopore alignment with ssDNA linker

FIG. 16 shows automatic individual DNA-nanopore alignment with flat bottomed micro-pillars

FIG. 17 shows modified ds λ-DNA to be used as the linker molecule

FIG. 18 shows the preparation process of ss λ-DNA to be used as the linker molecule

FIG. 19 shows the reaction that makes non-charged magnetic beads

FIG. 20 shows a single copy of DNA linked with a single linker molecule (with or without a bead) by limiting binding sites on the linkage hub: (a) two binding sites with a single linkage linker; (b) three binding sites with a dual linkage linker; and (c) four binding sites with a triple linkage linker

FIG. 21 shows double and single strand DNA (dsDNA and ssDNA) used as linker molecules, linking magnetic bead to single sequencing DNA through: (a) divalent streptavidin or other proteins with two binding sites, (b) antibody or other proteins with three binding sites, and (c) streptavidin or NeutrAvidin or avidin or other proteins with four binding sites

FIG. 22 shows DNA or linker molecules attach to a bead by emulsion droplet method

FIG. 23 shows magnetic control of DNA movement through nanopore

FIG. 24 shows magnetic control of DNA movement through nanopore with different linker molecules

### DETAILED DESCRIPTION

### Part I: Mechanical Control of DNA and Other Biological Molecules Passing Through Nanopores

This disclosure provides a method to precisely control the movement of a molecule, such as a DNA through a nanopore by a mechanical device, or more specifically, by a nanometer (nm) or sub-nanometer (sub-nm) precision piezo drive or combination of drives. In combination with electric force, it moves DNA into and out of nanopores either continuously or stepwise at a speed sufficient or slow enough for reliable base sensing depending on the base sensing method selected. For example; the ionic current blockage base sensing method using a protein nanopore requires at least 1 ms (millisecond) sensing time per base.

### The Basic Principle—Equilibrium of Forces

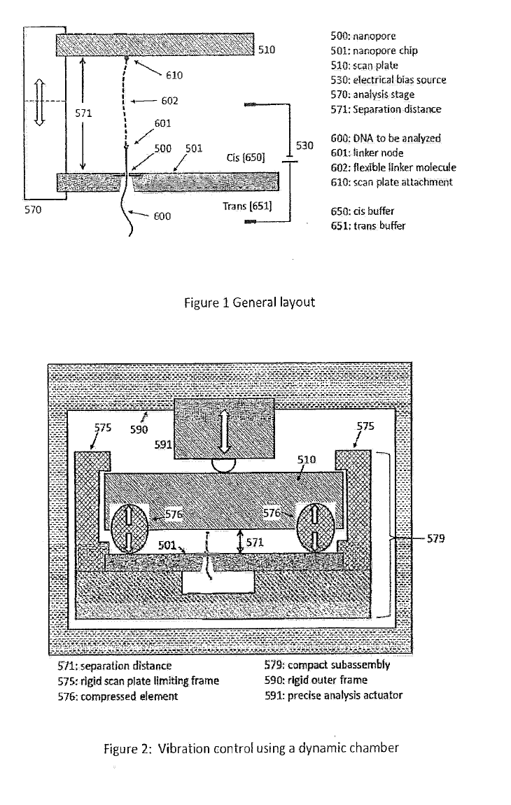

The basic principle is illustrated in FIG. 1 using a single strand DNA (ssDNA) as an example.

The nanopore (500) is part of a nanopore chip (501). The scan plate (510) is mounted parallel to the nanopore chip. Both the nanopore chip and the scan plate are coupled to the analysis stage (570) that can control their separation distance (571). The analysis stage is an appropriately selected automated precision linear motion stage. The nanopore chip mechanically divides the buffer volume into cis (650) and trans (651) buffer volumes on the opposing sides of the nanopore. The cis and trans buffer volumes are fluidically and electrically isolated by the nanopore substrate with communication through only the passage of the nanopore. An electrical bias source (530) maintains a voltage across the nanopore.

During the sequencing the DNA molecule being analyzed (600) spans the cis and trans buffer volumes by passing through the nanopore. On the cis side of the nanopore the DNA is joined to a flexible linker molecule (602) which is attached to a scan plate (510). The linker attachment to the DNA (i.e. the linker node) (601) and the attachment (610) of the linker to the scan plate are discussed further in a later section.

Since the DNA is negatively charged it is pulled through the nanopore to the trans side by the strong electric field inside the nanopore. When DNA on the cis side is depleted, both the DNA and flexible linker molecule are pulled taut between the scan plate and the nanopore. The linker and the DNA stretch somewhat under the tension and DNA bases continue to pass to the trans side of the nanopore until the tension of the stretched DNA matches the electric force acting on the DNA bases. This equilibrium tension is bias voltage dependent and corresponds to a specific stretched spacing of the DNA bases. If the nanopore and scan plate are moved closer together or moved further apart the tension in the DNA will also change (slightly) causing the DNA to move to restore the equilibrium condition. Provided that the separation speed is adequate and constant, the DNA will remain under constant tension as it moves. The DNA can either be pulled out of the nanopore, or inserted into the nanopore. DNA can therefore be sequenced in either direction without altering the nanopore bias voltage polarity or magnitude.

Maintaining a certain level of tension is required for accurate sequencing:
The speed of the DNA progression through the nanopore is better controlled when it is stretched under tension—when it is taut.
Keeping the DNA under a certain level of tension reduces the motion jitters due to Brownian motion. According to Lu et al (2011), the Brownian force is approximately in the order of 4 k_{B}T/a when DNA passes through nanopore freely. Here k_{B} is Boltzman Constant, T is buffer temperature in Kelvin, a is DNA base pair spacing. For ssDNA, a is about 0.7 nm when fully stretched. At room temperature (˜22° C.), the Brownian force can reach about 23 pN. However, when DNA is clamped at nanopore (stretched under tension) (Lu et al, 2015), the Brownian force is in the order of k_{B}T/Ls, where Ls is the Kuhn length of DNA. For ssDNA, Ls is about 1.5 nm. Hence, when ssDNA is stretched under tension with nanopore as lateral restriction, the Brownian force affecting base sensing is around 3 pN. In order to have accurate base sensing, the nanopore electric force (=DNA internal tension) should be much larger than 3 pN, for example 10 times greater. The greater the better but smaller than the binding force of DNA attachment.

### Motion Control Options and Considerations

As described above the controlled motion of DNA through a nanopore is determined by the separation distance (571) between a nanopore and a scan plate. Control of the separation distance is achieved by minimizing vibration between those elements and by implementing an analysis stage with sufficiently precise motion control.

Vibration control is achieved in several ways:
The mechanical path between the nanopore and the scan plate should be as short as possible. A compact assembly can be utilized.
The mechanical connection between the nanopore and the scan plate should be as stiff or rigid as possible. This means making components out of stiff materials and thick.
The mechanism should be isolated from external vibrations and noise. Standard passive and active anti-vibration methods can be employed similar to what is used in atomic force microscopy and interferometry.

The analysis stage (570) has some requirements:
The actuation must be stiff or rigid—it cannot compromise the vibration control measures listed above.
It must have sufficient precision—such as with sub-nanometer (sub-nm) resolution from about 0.1 nm to about 1 nm for DNA base sensing, or with nanometer resolution from 1 nm to 10 nm for molecules with larger base units spacing distances.

A mechanism that meets these specifications is a piezo-electric drive (alternately called a piezo-electric stage, piezo-electric stack, piezo-electric actuator or piezo-electric transducer). Piezo-electric devices are extremely stiff, have the necessary precision, and move without vibration.

Alternative mechanisms may also be suitable. Some possible options include less precise actuators combined with mechanical reduction (i.e. leverage) to achieve the necessary precision. Conceivably a servo driven actuator could work if vibration can be minimized. Piero-electric linear motors may also work, if they are controlled to eliminate vibration.

The degree of precision required in the stage motion is certainly attainable with present day technology. Examples of nanometer precision piezo electric drives can be found in scanning tunneling microscopy (STM) and atomic force microscopy (AFM), which are widely used in biological microstructure analyses. King and Golovchenko (2005) demonstrated that they could thread a 105 nm long 5 nm diameter nanotube through a nanopore using an AFM tip. They could move the nanotube in and out the nanopore at a speed around 800 nm/s and measure the ionic current changes. Considering a unit length of 0.7 nm for the single strand DNA, it is equivalent to about 1 ms per base.

Another example of commercially available nanometer precision piezo drives is the P-62x series precision Z-stages from Physik Instrument GmbH & Co., which have 0.1 to 1 nm precision with positioning accuracy 0.02%, traveling range 50 to 400 μm. With the right load and operating frequency, it can easily achieve speeds from 1 ms to 10 ms per base, slow enough for base sensing yet fast enough for rapid DNA sequencing. For example, a 100 kb DNA strand, can be sequenced in about 8 minutes with throughput of 5 ms per base.

### Dynamic Chamber—Minimizing the Mechanical Path Length

One approach to minimizing vibration is to assemble the nanopore and the scan plate into a compact subassembly (579) such as is depicted schematically in FIG. 2. The scan plate (510) and the nanopore chip (501) are held apart by the elastic outward force (indicated by large vertical arrows) of a compressed element (576) which pushes them apart. The compressed element (576) may also provide a liquid seal around the chamber or an independent interior seal could be utilized. If an independent seal is included, then the compressed element (576) could be a non-sealing type such as a wave spring or a flexure mount.

In the layout shown the nanopore chip is bonded to a rigid frame (575) that constrains the motion of the scan plate and determines its relaxed position (i.e. the maximum separation from the nanopore chip). The scan plate can be moved toward the nanopore by squeezing the subassembly (579) together using the analysis actuator (591) which is coupled to a rigid frame (590) that wraps around the subassembly (579)

This design shortens the mechanical path by bridging the gap between the scan plate and the nanopore chip. Although the compressive element (576) can be squeezed vertically it can be chosen such that substantial force is required such that it acts as a strong coupler. The compressive element may also be strongly non-isotropic meaning that although it can be squeezed vertically it may strongly resist lateral shearing deformation (i.e. it may be very stiff laterally). The compressive element can also have dampening properties or be mounted in parallel to a dampening element that further reduces vibration.

FIG. 2 further illustrates the main principles of bundling the scan plate and the nanopore in a compact subassembly. This concept can be implemented in numerous ways;

One embodiment discloses a subassembly in which the scan plate and the nanopore are pressed together or in near proximity to each other when the subassembly is at rest and are pulled further apart when the subassembly is acted on by an external analysis actuator. That implementation may be advantageous because it allows the subassembly to be fabricated with the scan plate and the nanopore chip to be substantially parallel and separated by a predetermined minimum distance (e.g. use micrometer shims or patches) when the subassembly is in its rest state (i.e. not acted on externally). Procedurally this is convenient for sequencing. It is discussed in a later section.

It is important to consider “compression set” or more generally “non-elastic deformation” when creating this subassembly. In some embodiments, deformable materials such as rubber with restorative force (i.e. elastic force) may be diminished if they are compressed or stretched for a prolonged time. To manage the effect of “set” it is best to design the subassembly such that during operating conditions the moving elements are further from equilibrium than it during storage. Alternatively, a material that doesn't degrade such as spring steel can be chosen.

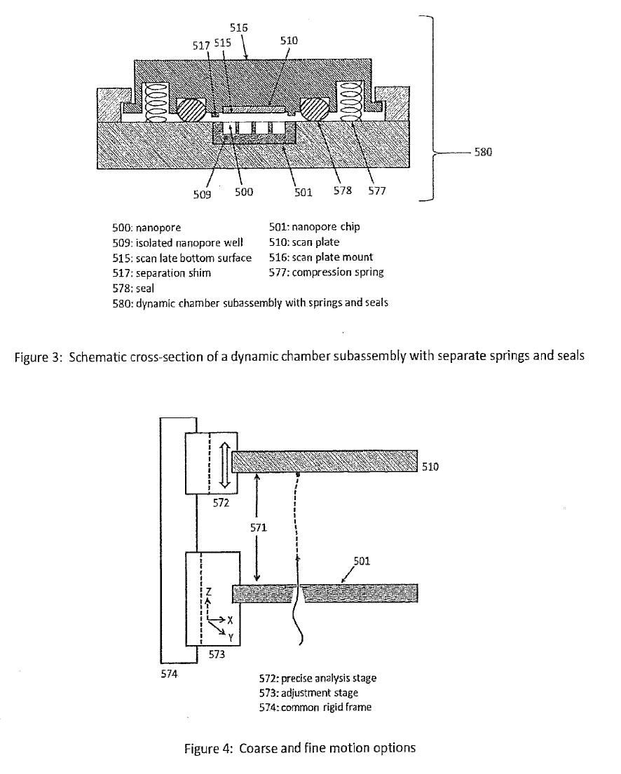

In FIG. 2 the compressive element is shown acting both as a spring and also as a seal confining the cis buffer above the nanopore. These functions can be separated for example with an independent flexible seal forming the perimeter walls of a chamber between the nanopore chip (501) and the scan plate (510) and separate compressed springs outside the chamber providing the outward force (see FIG. 3). Elements that provide restorative force when extended outward from a rest position such as extension springs can be used. Elements that provide restorative force when bent from a rest position leaf springs can be used. Seals that stretch under tension or bending may also provide restorative forces. FIG. 3 provides one of the alternatives. The dynamic chamber subassembly illustrated in FIG. 3 has separate sealing elements (578) and spring elements (577). By splitting these functions the spring force can be substantially increased without risking loss of elasticity in a compressed element, i.e. non elastic deformation/compression set. The bottom surface (515) of the scan plate (510) is recessed with respect to the bottom surface of the separation shim (517) of the scan plate mount (516). This prevents contact with the nanopore chip (501). In certain embodiments, each nanopore (500) has an isolated well (509) on the trans side.

In both FIGS. 2 and 3, inlets and outlets for fluid communication or buffer exchange with external fluid reservoirs are not illustrated but they can be easily achieved by adding a passage or port on either the nanopore chip subassembly or the scan plate subassembly.

### Additional Stages for Alignment and Scanning

The scan plate and the nanopore chip may require adjustment of their relative position in addition to the analysis-stage/analysis-actuator motion required for DNA sequencing. These adjustments may be required for sample loading, or for extending the motion of the analysis stage. They may also be required to implement certain sequencing protocols described in a later section.

FIG. 4 illustrates the basic idea of including an adjustment stage (573) in addition to the precision analysis stage (572) controlling the separation distance (571). These stages are coupled rigidly to the scan plate (510) and to the nanopore chip (501). They are each coupled to a common rigid frame (574). It should be noted that this diagram merely illustrates the mounting options of the stages and the frame. An alternative embodiment would encompass a functional design that would avoid the cantilevered mounting arrangement of the scan plate and the nanopore chip which would minimize vibrations.

Additional stage(s) used for adjustment etc. do not necessarily require the same precision as the analysis stage. Stages with coarser precision motion (i.e. μm precision) may be sufficient and may offer longer ranges of motion. These stages do not need to be implemented together as shown in the figure. For example, the X and Y axis could be implemented to move the nanopore chip (501) with respect to the rigid frame (574) and the Z axis could be implemented to move the analysis stage (and therefore the scan plate) with respect to the rigid frame (574). Also, since it is only the relative separation of the scan plate and the nanopore chip that is essential, the analysis stage could be implemented on the nanopore chip as well.

It should also be noted that although the illustrations indicate the nanopore chip surface oriented horizontally this convention is used for ease of understanding and is not a requirement. Some embodiments may orient the nanopore chip surface and the scan plate vertically, at an intermediate oblique angle, or even inverted and orient the stage motions accordingly.

The above paragraphs explain the flexibility in adjustment of the relative positions of scan plate and the nanopore chip. It is important to remember that the mechanical stiffness/rigidity of the assembly should not be compromised. It is possible to eliminate unnecessary adjustment stages that would otherwise extend the mechanical path from the nanopore chip to the scan plate.

### Position Calibration

At times during the alignment procedure the scan plate must be brought in close proximity to the nanopore chip. Prior to performing a sequencing procedure it may be necessary to calibrate the position of the analysis stage or actuator such that the minimum separation distance and the coordinates are known, i.e. the numerical position in the positioning system driver that corresponds to closest contact between the scan plate and the nanopore chip surface. The position should be known with an accuracy of a few microns.
Calibration can be performed by several means:
Optical—This type of calibration requires a window or an optical path that allows the gap between the scan plate and the nanopore to be observed and measured.
Electrical Contact—This type of calibration requires that the scan plate and the nanopore chip be filled with fluid, and it would require some portion of each of the scan plate and the nanopore chip to be made of conductive materials. These conductors could interfere with sequencing measurements. If the manufacturing is sufficiently precise the electrical contact point or points could be made at locations that are sealed away from the buffer solution.
Force—The analysis stage slowly brings the scan plate and the nanopore chip closer together. A sensitive force transducer determines when they make contact. Alternatively, raised features of known height on either the scan plate or the nanopore chip or both make the first contact to avoid possible damage to the DNA plate or the nanopore chip. This is a practical solution since the force sensor can be added to the mechanical actuator and no further changes are required to the plate or nanopore or access requirements. The force sensor layout can be designed such that minimal force is applied to the nanopore chip and the scan plate.
Separation Feedback—Assuming that the nanopore chip and the scan plate are pushed close together in a subassembly without surface contact such as the “raised features” described above. This could be achieved by a passive spring element in the assembly, such as in FIGS. 2 and 3. The analysis stage could be configured to pull apart a subassembly in which the nanopore chip and the scanning plate are pressed together in a relaxed state. Position feedback could be generated from the base-sensing at some step of the sequencing procedure. This would be considered “functional feedback” since it indicates the analysis stage position necessary to begin pulling DNA out of the nanopore. Electrical contact or force measurements could also be used as feedback of how the analysis stage contacts the subassembly.

### Sensor Geometry and Arrays

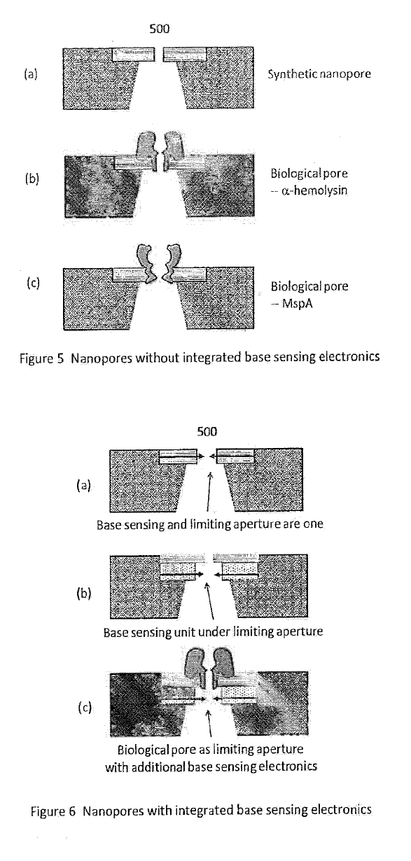

The methods described in this disclosure for precise mechanical control of DNA translocation through nanopores apply to any types of nanopores without limitation, either synthetic nanopores (FIG. 5a, such as Si₃N₄ pores, graphene pores, MoS2 (molybdenum disulphide) pores and any other solid state nanopores), or biological nanopores (FIGS. 5, b&c, such as α-hemolysin pore and MspA pore), or even combination of synthetic and biological pores. The nanopores can have integrated base sensing apparatus with either synthetic pores (FIGS. 6 a&b) or biological pores (FIG. 6c). The base sensing apparatus is either within the substrate (FIG. 6a) at the nanopore or peripheral to the nanopore (FIG. 6b).

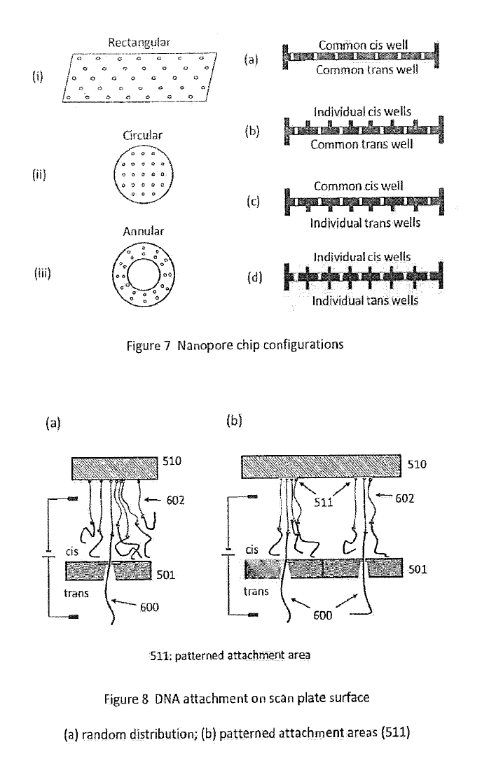

The nanopore chip mentioned in this disclosure may contain a single pore or a plurality of pores or an array of pores. The pattern of nanopores on the chip can be in any shape, such as square, rectangular, linear, elongated rectangle, split rectangle, circular, elliptic or annular see FIG. 7 (i) to (iii). Suitable patterns will be dictated by practical considerations such as manufacturing methods, fluid flow and electrical connections from the perimeter.

Certain base sensing methods such as ionic current sensing require electrical measurements that span the nanopore (i.e. one electrode is on the trans side of the nanopore, one electrode is on the cis side of the nanopore). To make these measurements for a plurality of nanopores it may be necessary to electrically isolate the buffer surrounding the electrical contact on at least one side of each nanopore from all the other nanopores. These various permutations of common and isolated wells on either side of the nanopore are illustrated in FIG. 7. If electrical isolation is a requirement for the base sensing method then a possible arrangement is to have a common cis well and isolated trans wells containing isolated buffer and sensing electrodes (FIG. 7c). This could be manufactured with the isolated trans wells filled with buffer solution and the sample DNA introduced from the common cis well.

### Patterned DNA Attachment

In an embodiment, DNA molecules can be attached to the scan plate with or without a linker molecule. The simplest way is to attach DNA onto the scan plate surface randomly (FIG. 8a), then move the scan plate or nanopore substrate laterally in an area-scan pattern for repeated sequencing. The nanopores on the nanopore chip cannot be arranged too close due to the separation requirements of buffer well fabrication and the electric measurement. The nanopores should be sufficiently spaced relative to each other such that they do not interfere with each other. In an embodiment, the nanopore spacing is over 100 μm or even over 1 mm, with a scan pitch of several μm or less per sequencing cycle it may take too much time to scan through the space between nanopores in order to get all target DNA molecules sequenced. As a solution to this problem, the DNA molecules can be attached only to very small patterned areas (511), e.g. several μm or less, corresponding to each nanopore on the nanopore array chip, see FIG. 8b. This way, the scan area is reduced and the sequencing of all or most of the DNA molecules can be done in a shorter time. The patterned attachment may require precise alignment (μm accuracy) between the patterned areas and the nanopores. This can be easily done with present manufacturing and instrumentation.

### Micro-Pillars—If Necessary

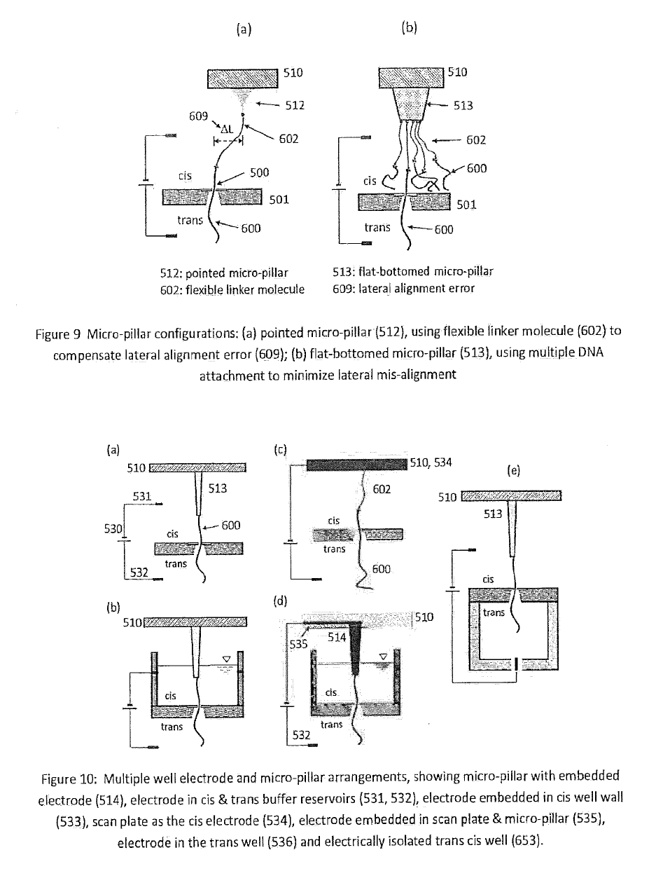

Although utilizing a flat scan plate is an exemplary embodiment, some configurations of the wells and of the DNA attachment require micro-pillars to be included on the scan plate (FIGS. 9 & 10). Micro-pillars (512, 513) are extensions that protrude from the scan plate and which match the spacing and lateral arrangement (pattern) of the nanopores. They can be made by attachment or formed as a continuous part of the scan plate or by various microfabrication means. Micro-pillars extend the attachment (or contact) location of the DNA outward from the scan plate. This is done for several reasons:
The micro-pillar can provide a larger fluid flow path by adding gaps above the DNA attachment site. This can be important when the scan plate and nanopore chip are brought close together and the space between the DNA attachment site and the nanopore chip is constricted. Increasing the fluid path reduces the fluid flow velocity and reduces pressure build up during motion at close separation distances.
The micro-pillar provides better fluid contact with the DNA attachment site. If the tip of the micro-pillar is relatively narrow the DNA attachment site will experience more flow. The micro-pillar also adds an obstacle to lateral flow which helps promote mixing.
Placing the DNA attachment on the tip of the micro-pillar allows for a more concentrated attachment of DNA. This can make the sequencing process more efficient since it may allow the DNA to engage with the nanopore more readily.
If isolation wells are included on the cis side of the nanopore the micro-pillars can extend the DNA attachment into buffer filled individual wells while maintaining the electrical isolation (FIGS. 10b & 10d). This requires that each micro-pillar is electrically isolated from the other micro-pillar. This might be combined with an electrical probe placed inside the micro-pillar for sensing DNA moving through the corresponding nanopore such as illustrated in FIG. 10d (514, 535). In these arrangements the cis side of the nanopore array is divided into electrically isolated wells (653).

The micro-pillars can be short or tall depending on the required function. The attachment surface can be rounded or flat depending on method of attachment. The different methods for attachment of the DNA are discuss in a later section.

Micro-pillars require precise alignment to the corresponding nanopores. This can be accomplished with a precisely manufactured subassembly or by using motorize lateral adjustment drives combined with feedback from the nanopore sensors.

The number of strands of DNA that are attached to the micro-pillar is largely determined by the manufacturing method that determines the area for DNA attachment. This area can be sized for any number of DNA stands from a single strand to hundreds or more depending of the sequencing method desired. The density of DNA is also influenced by the concentration of the DNA in solution when the attachment is made.

### Preparation and Sequencing Procedures

### Overview

The sequencing procedure can be divided into two phases for the purpose of discussion:
Engaging DNA: Getting the free end of DNA sufficiently close to the nanopore such that it can be engaged or inserted in the nanopore (e.g., drawn into the nanopore by the electric force)
Sequence: Slowly drawing the DNA through the nanopore while recording from a sensor. Reversing direction and repeating as necessary.

The Engaging DNA phase has a few significant variations depending on the geometry of the scan plate, the spatial distribution of the DNA on the scan plate, the presence or absence of a flexible linker molecule between the DNA and its attachment to the scan plate or micro-pillar.

### Engaging DNA—Use of a Flexible Linker

The DNA can be lengthened by adding a flexible linker molecule to one end and attaching the linker molecule to the scan plate or to the micro-pillar. This embodiment allows some degree of misalignment between the attachment position on the scan plate and the nanopore position while sequencing the entire length of the DNA. It also allows the DNA to engage with the nanopore while the scan plate and the nanopore chip are not touching.

FIG. 1 illustrates the linker molecule in relation to the scan plate and nanopore chip. The linker molecule (602) can be any linear molecules or molecular complex, such as cellulose fibers, long polypeptide chains or oligonucleotides or bacterial/viral DNA/RNA, or linear polymer chains. An embodiment has a length of about 10 to about 50 micrometers (μm). Another exemplary length is about 5 to about 100 μm. Yet another exemplary length is about 5 to about 1000 μm. The λ-DNA (isolated from bacteriophage lambda) is an example for a linker molecule. It has 48.5 kilobases. When fully extended (drawn straight without stretching), it is about 34 um long (single strand) or 16 um long (double strand), a perfect length to be used as the linker molecule.

The scan attachment (610) is the attachment of the DNA or the linker molecule either directly to the scan plate (510) or to the micro-pillar.

The linker node (601) is the location of the connection between the DNA strand (600) and the flexible linker molecule (602). It can be a protein, such as Streptavidin or Neutravidin or antibody, or a non-magnetic bead or just simply a chemical bond, such as phosphodiester bond when the target DNA is ligated to a linker λ-DNA.

Although a permanent attachment is acceptable, the DNA/linker attachment to the scan plate (610) can also be a reversible non-covalent bond such as biotin-streptavidin coupling or a covalent bond breakable under certain buffer conditions or catalysts so that the sequencing can be repeated by detaching the sequenced DNA and re-attaching a new DNA. The biotin-streptavidin bond is very strong, having enough strength to hold the DNA onto the scan plate during sequencing, however, it is reversible by incubating a short time in non-ionic aqueous solutions at temperatures above 70° C. (Holmberg et al, 2005). Covalent bond is usually much stronger than non-covalent bond. An example of reversible covalent bond is the amide bond formed between a carboxylic acid and an amine. For the attachment of DNA, one can functionalize the scan plate surface with carboxylic acid and attach amine-modified DNA or linker molecule to the surface through an amide bond. The amide bond can be broken by hydrolysis under strong acid (such as HCl) or strong base (such as NaOH) when the sequenced DNA needs to be removed later. The ester bond formed between a carboxyl group and a hydroxyl group behaves the same, which can be broken by strong acids and bases as well as temperatures. Other methods regarding DNA or protein attachment to solid surfaces, either metal, glass, or plastic can be found in the review articles of Singh et al (2011) and Sassolas et al (2008), and in the articles of Liu and Rauch (2003) and Fixe et al (2004), as well as on the websites of some biotechnology companies, such as ThermoFisher Scientific and Integrated DNA technologies.

### Engaging DNA—Lateral Alignment to Match the DNA Deposition Pattern

The scan plate and nanopore chip surfaces are substantially parallel to each other. These DNA attachment surfaces (i.e. the scan plate, the surfaces of the blunt micro-pillar, or tips of pointed micro-pillar) must be brought close together, typically within a few microns, for the DNA to have sufficient reach to engage in the nanopores. The DNA can be attached to the scan plate in various patterns for reasons of optimization etc. These layouts dictate the required lateral positioning of the scan plate with respect to the nanopore chip: (FIGS. 8 & 9 illustrate some of these layouts)
1) If pointed micro-pillars are used (512), the tips of the micro-pillars must be laterally positioned to match the nanopore array positions within a reasonable error tolerance, which for some embodiments would usually be from a few nanometers to several micrometers. The required tolerance is dictated by the length of the flexible linkers (602) which can compensate for any lateral misalignment (609). If the DNA is directly attached to the scan plate without a linker then the lateral misalignment is dictated by how long a length of the sample DNA is acceptable to be omitted from sequencing (FIG. 9a) For long DNA fragments it may be acceptable to omit several thousand bases.
2) The DNA attachment can be restricted to a pattern of locations on the scan plate matching the nanopore pattern on the nanopore chip (FIG. 8b). For this “matching pattern” approach the lateral position must align that DNA pattern to the corresponding nanopore pattern e within the limits that the nanopore can be reached by the flexible linker. A similar close alignment must take place if the DNA is attached to the scan plate using flat-bottomed micro-pillars (513) as in FIG. 9b.
3) The DNA attachments can be made uniformly across the scan plate (FIG. 8a). This can be a useful approach for scanning all the DNA and minimizing repeat scanning. The DNA at the location directly aligned with the nanopore always has the greatest probability of being engaged in the nanopore since it is the closest. If after each DNA sequence is recorded the alignment of the scan plate and the nanopore chip can shift to a new location, then the DNA at that new location is favored to enter the nanopore. By systematically repositioning using the X and Y lateral adjustments a small area of X and Y offsets can be covered. By sampling an area with dimensions corresponding to the nanopore array lateral spacing all the DNA on the scan plate can be sequenced with equal probability.

### Sequencing—Buffer Conditions, Bias

In an embodiment, the cis and trans reservoirs (650 and 651) are generally filled with electrically conductive salt buffer (e.g. 1M KCl 10 mM Hepes buffer, pH8), see FIG. 1. The type of buffer to be used really depends on the molecule to be analyzed, the pore surface chemistry and the base sensing requirements. In some embodiment, the trans reservoir may need to be filled with a different buffer with different conditions or compositions, such as different pH or salt concentration, addition of EDTA or minerals, surfactants, etc. In some embodiment, the trans reservoir may need to be filled with a gel or other substances that allows the target molecule to move through without being chemically modified. The nanopore bias voltage (530) is applied through two Ag/AgCl electrodes placed in the cis and trans reservoirs across the nanopore chip (501). Some base sensing methods require that at least one of these electrodes is specific to each nanopore if the nanopore chip has a plurality of nanopores. The buffer has sufficient ionic content to be conductive, but to some extent the electric fields from the nanopore voltage bias extend outside the nanopore to pull at the charged DNA nearby.

Once the target DNA fragment(s) is engaged within the nanopore(s) it can be translocated in and out as previously described. The DNA (600) can be sequenced as it is translocated through the nanopore and recorded with an appropriate base sensing method, such as ionic current blockage, recognition tunneling or Nanowire FET.

### Sequencing—Piecewise Repeated Sequencing Scheme

In some embodiments, the DNA may need to be sequenced multiple times in order to achieve the required accuracy. A common scheme is to record the base sensing of the entire DNA by pulling the DNA completely out of and then insert it back into the nanopore several times. For an array of nanopores, numerous DNA fragments of different, lengths need to be sequenced at the same time. Some DNA fragments will finish much sooner than others during each sequencing run. Sometimes, during the insertion process, some DNAs may not be able to get into the pore and miss the repeat of sequencing.

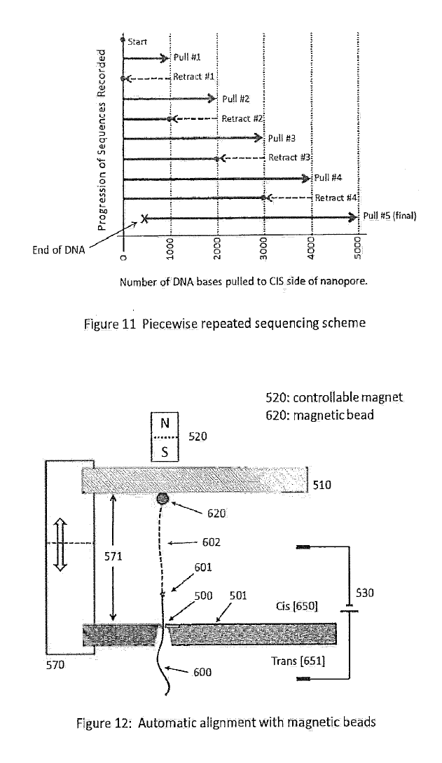

Here, we introduce a piecewise repeated sequencing scheme that guarantees each DNA fragment will receive the same number of repeat recordings, which is illustrated in FIG. 11. The procedure is:
1. Start with the DNA engaged and fully inserted into the nanopore;
2. Pull the first portion of the DNA to be repeated, e.g. 1000 bases, out of the nanopore;
3. Insert the 1000 bases back into the nanopore completely;
4. Pull 2000 bases of the DNA out. The first 1000 bases have passed the pore sensing point three times already, while the second 1000 is a new section to be repeated.
5. Insert the new 1000 base section back into the pore and then pull it out with additional 1000 bases.

Continue repeating step 5. As illustrated in FIG. 11, the DNA is progressively pulled out to base positions 1000, 2000, 3000, 4000 etc. The procedure continues until the entire DNA is pulled out of the nanopore and therefore becomes disengaged. In the example (FIG. 11) the DNA sample is 4600 bases long and the procedure terminates as the DNA is pulled to position 5000.

The base-sensing can be recorded both ways while the DNA is being pulled out and while it is being inserted into the pore. It may be more convenient for data analysis to record only while pulling the DNA out. The same base sensing method can be used in both directions, or a different method could be used during the insertion. A faster or slower speed may be used during the insertion if this results in a useful change in the base-sensing contrast or simply to perform the insertion more rapidly without base sensing.

In the example, each section of the DNA is sequenced three times. When a DNA fragment is completely pulled out of the pore, only the last partial section is sequenced a single time. The majority of the DNA fragment is sequenced three times. If more repeats are needed, the pulling and insertion lengths can be adjusted to record each section more times. It may be advantageous to insert several extra bases to such that adjacent sections overlap to insure sufficient repeat recording of the bases at the boundaries.

### Part II: Automatic Alignment of DNA and Other Molecules with Nanopores

This disclosure also provides a method for automatic alignment of DNA with nanopores by using an adjustable magnet and magnetic beads. Aligning the DNA means to pull the DNA in a substantially straight line manner away from the nanopore, normal to the surface of the nanopore chip, and in line with the motion direction of the analysis stage. This places the end of the DNA (or places the end of the attached linker) against the scan plate at a location directly in line with the nanopore in the direction of the analysis stage motion such that it is pulled straight away from the nanopore. This alignment of the DNA insures that the length of the stretched DNA passing through the nanopore is exactly equal to the distance the stage moves, either in or out. If the attachment location is misaligned, as in the case of randomly attached DNA, the speed changes as a function of the separation distance and the force required to bond or hold the DNA against the scan plate is greater.

### The Magnet

The purpose of the magnet is to pull the end of the DNA or linker that is on the cis side of the nanopore toward the scan plate. The magnetic field must therefore be effective in the space between the nanopore chip and the scan plate. The force of a magnet acting on a volume of magnetic material such as a magnetic bead, \vec{Fₘ} is roughly proportional to the gradient of the magnetic field \vec{H₀}, the magnetization of the material \vec{M}, and the volume of the magnetic material in the bead V_{BEAD} as given by the formulas below.

\vec{Fₘ}=μ₀*V_{BEAD}*(\vec{M}·∇)\vec{H₀} (complete formula)

For strong magnetic fields the magnetization of the magnetic beads approaches its saturation limit and is no longer proportional to the driving magnetic field. The force formula can be simplified to:

\vec{Fₘ}≈V_{BEAD}*M_{Saturation}*\vec{G} (simplified formula for saturated magnetic materials)

### Where:

M_{SATURATION} is the bead magnetization at saturation
\vec{G} is the gradient of the magnetic field.

In practical application this means the magnetic field must be strong in the gap between the scan plate and the nanopore chip to saturate the magnetic bead magnetization and the field must have a strong gradient increasing toward the scan plate.

This field condition can be achieved by placing a magnet directly behind the scan plate with a pole facing toward the nanopore chip as illustrated in FIG. 12. The magnet can be either an electromagnet or a permanent magnet. Electro-magnets can easily be controlled by varying the driving current. The field of a permanent magnet in the gap can also be increased or decreased simply by moving the permanent magnet closer or further away from the scan plate. Permanent magnets can have very strong magnetic fields, particular neodymium magnets (NdFeB magnets). Both the field strength and the field gradient can be enhanced by arranging multiple permanent magnets to shape and reinforce the magnetic field such as in a Halbach array.

### Magnetic Beads

Although there are several variations for the alignment procedure, they all utilize some variation of magnetic beads. The beads can be super-paramagnetic, i.e. they can be magnetized in the presence of a magnetic field but they will lose their magnetism when the magnetic field is removed. They should be as small and as light as possible to avoid hindering the DNA movement toward the nanopore but sufficiently large to prevent the bead from entering the nanopore and have enough magnetic content to generate sufficient magnetic force to hold and move the target DNA molecule. In one embodiment, the size range is about 200 nm to 3 μm. In another embodiment, the size range is about 50 nm to about 10 μm. In some embodiments, the size range may be about 10 nm to 50 μm. The required bead size is dictated by the amount of force required. Flexible linker molecules can be attached to the magnetic beads via covalent bonds or non-covalent bonds, similar to the attachment of the DNA/linker molecules to the scan plate described in Part I.

### Variations

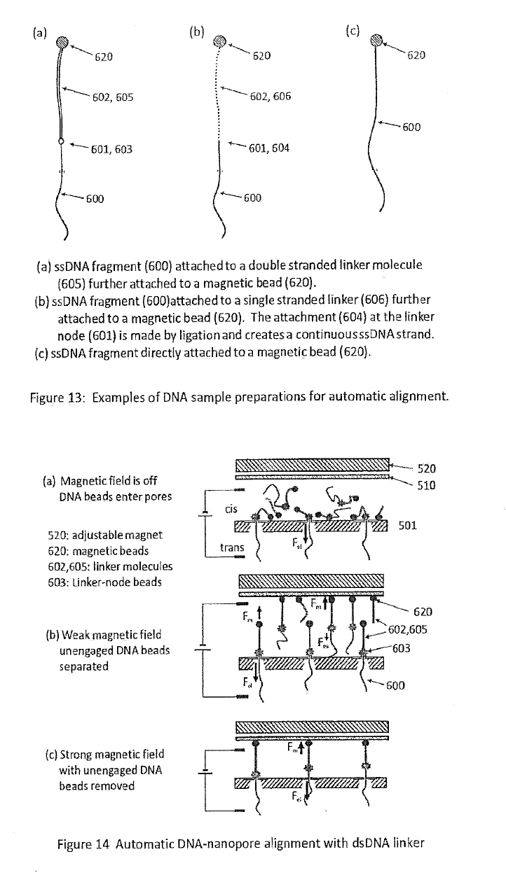

The alignment can be performed with several different preparations of DNA and magnetic beads: These preparations are illustrated in FIG. 13. In this figure, we assume a λ-DNA (single strand or double strand) or similar linear polymer is used as the linker molecule.

### Method 1: Alignment Using a Stopped Linker

A flexible linker molecule (605) is attached to the magnetic bead (620) at one end and is linked to the sample DNA (600) through an enlarged linker node (601) which is larger than the entrance of the nanopore and functions as a brake or stopper when the DNA translocates through the nanopore (FIG. 13a). The linker node refers to a protein or other means (603) by which the flexible linker molecule can be attached to the sample DNA. The linker node can be a non-magnetic bead or a large protein complex (such as antibody, NeutrAvidin, streptavidin, or avidin), or a large polymer ball, or other molecules, preferably non-charged or weakly charged in sequencing buffer. In some embodiments, it should be at least twice as large as the nanopore entrance.

The DNA is tagged with the magnetic beads as follows:

[magnetic bead]+[flexible linker molecule]+[linker node]+[optional calibration DNA oligo]+[ssDNA sample]

A specific example of this is a magnetic bead tagged DNA assembly below which uses a double stranded λ-DNA as the flexible linker. The double stranded λ-DNA is about 50 kilobases and has an unstretched length of about 16 μm.

[1 μm DynaBead® MyOne™ Carboxylic Acid bead]+[amine & biotin modified ds λ-DNA]+[NeutrAvidin]+[biotinylated ssDNA sample]

In one embodiment, the amine & biotin modified ds λ-DNA is shown in FIG. 17. One end of the ds λ-DNA is added an amine linker on both strands, facilitating the amide bond formation with the carboxylic acid modified magnetic bead surface. The two amine linkers, each having a carbon chain of different length (C6 & C3) (a) or of the same length (C6 & C6) (b), provide a flexible free end for amide bond formation on both λ-DNA strands. The other end of the ds λ-DNA is biotinylated with two equal flexible free tails (can be made unequal too) to be attached to the NeutrAvidin, which has four biotin binding sites. To have both strands attached increases the strength of the linker molecule.

Alternatively, an amine functionalized bead can be used with a crosslinker, such as BS3 crosslinker, to bind the amine modified ds λ-DNA to replace the carboxylic acid functionalized bead for a neutral charge bead surface.

FIG. 14 illustrates a simplified physical layout showing the nanopore chip (501), the scan plate (510), the magnet (520) and tagged DNA suspended in buffer solution.

The alignment procedure is as follows:
1. The chamber on the cis side of the nanopore array is filled with a buffer solution containing the magnetic bead tagged DNA (as described above).
2. The nanopore bias voltage is set to draw the negatively charged DNA into the nanopore. (FIG. 14a) The free end of the DNA (600) enters the nanopore (500). This can be detected by monitoring the nanopore open pore current. The progress of the DNA molecule into the nanopore is stopped by the linker node which cannot enter the nanopore. A properly chosen linker node can also block other DNA from entering the nanopore.
3. Some tagged DNA samples will not occupy a nanopore and stay scattered on the nanopore chip surface or in the cis buffer reservoir. We term the tagged DNA inside a nanopore as “engaged” while the tagged DNA outside the pore “unengaged”. It may be desirable to remove the unengaged DNA from the cis reservoir during the sequencing of the engaged DNA if interference is a concern. This can be accomplished by a wash step performed before the magnetic field is engaged. The unused DNA can be sequestered in a storage reservoir and may be retained or released by a switchable magnet for next round of sequencing process.
4. The scan plate (510) is brought relatively close to the nanopore surface, but with the gap is sufficiently larger than the longest linker molecule length. Assume Fₘ is the magnetic force acting on the magnetic beads, Fₑₛ the electric force acting on the unengaged DNA, and Fₑₗ the electric force acting on the engaged DNA. Since Fₑₗ is several orders of magnitude larger than Fₑₛ, by activating the adjustable magnet (520), starting with a zero or very weak magnetic field, and gradually increasing the magnetic field strength such that Fₑₛ<<Fₘ<<Fₑₗ (<< means far less than) (FIG. 14b). The magnetic force is sufficient to draw any remaining unengaged DNA to the scan plate, but insufficient to pull engaged DNA out of the nanopores where they are held by very strong electric force. The magnetic beads (620) linked to the engaged DNA will float straight above the linker-node beads (603), aligning perfectly with the DNA and therefore aligning perfectly with the corresponding nanopore (see FIG. 14b).
5. The scan plate is then lowered further to allow all magnetic beads corresponding to the engaged DNA to contact the scan plate directly above them. After all the magnetic beads (620) are in contact, the magnetic field is increased to make the magnetic force Fₘ much larger than the electric force Fₑₗ on the engaged DNA (Fₘ>>Fₑₗ). The magnetic beads (620) are pulled tightly against the scan plate (510), so that they track the motion of the scan plate (510) precisely (FIG. 14c). The DNA can be sequenced as it goes in and out of the pore as many times as required by moving the scan plate (or nanopore substrate) in a precisely controlled speed. After the DNA is sequenced, the magnetic force can be turned off and on to release and remix or circulate the tagged DNA and start another round of sequencing in a short time without changing buffer or additional sample processing. Note that in some situation some magnetic beads may have residual magnetization and remain attached to the scan plate surface after the magnetic field is switched off, a secondary adjustable magnet may be placed on the trans side of the nanopore pore to pull the magnetic beads off. The secondary magnet can be much weaker than the main magnet. Alternatively, other means can be used to remove the loosely attached magnetic beads, such as dielectric force, fluid shear force, etc.

6. Alternatively, the magnetic beads can be chemically bonded to the scan plate at the initial contact to guarantee a firm contact between the bead and the scan plate surface so that the bead therefore the linker molecule and the attached DNA can be moved precisely with the scan plate movement. This may be needed in some special cases where the required maximum magnetic force is compromised, e.g. if smaller magnetic beads have to be used.

### Method 2: Alignment Using a Single Stranded DNA as the Linker

The magnetic bead (620) is attached by a flexible linker molecule (606) which is linked to the sample DNA (600) by ligation or other means (FIG. 13b). The flexible linker molecule is of the same type as the sample DNA and the attachment is such that the result is a single continuous single stranded molecule. The entire continuous molecule can therefore pass through the nanopore without an interruption at the transition between the linker and the sample DNA.

The tagged DNA is constructed as follows:

[magnetic bead]+[flexible linker molecule][ligation][DNA sample]

A specific example of this is the magnetic bead tagged DNA assembly below which uses a single stranded λ-DNA (ss λ-DNA) as the flexible linker. The ss λ-DNA is about 50 kilobases and has a length of about 34 um when fully stretched.

[1 μm DynaBead® MyOne™ Carboxylic Acid bead]+[amine modified ss λ-DNA]+[end modified ssDNA sample]

Briefly, the steps involved in the sample processing are shown in FIG. 18. The sample will be stored in the form of ds λ-DNA to avoid possible hairpin formation or cross link between different DNA fragments. The unwanted strand of the ds λ-DNA will be denatured and removed before use.

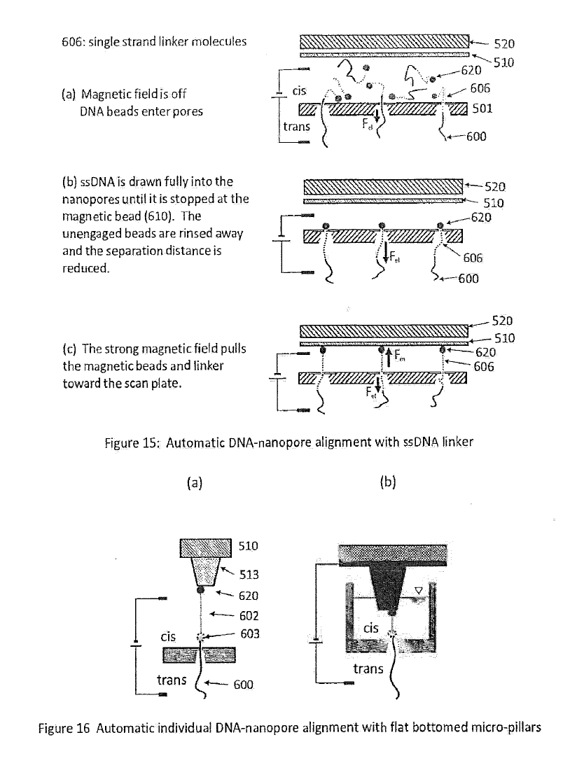

The alignment procedure is as follows:
1. The chamber on the cis side of the nanopore array is filled with a buffer solution containing with the magnetic bead tagged DNA (as described above).
2. The nanopore bias voltage is set to draw the sample DNA (600) into the nanopore (FIG. 15a). The free end of the DNA enters the nanopore. This can be detected by monitoring the nanopore open pore current. The progress of the DNA molecule into the nanopore is only stopped at the magnetic bead (620) which cannot pass through the nanopore. Both the ssDNA sample (600) and almost the entire flexible linker (606) pass through the nanopore to the trans side (FIG. 15b)
3. Some tagged DNA samples will not occupy a nanopore and stay scattered on the nanopore chip surface or in the cis buffer reservoir. We term the tagged DNA inside a nanopore as “engaged” while the tagged DNA outside the pore “unengaged”. It may be desirable to remove the unengaged DNA from the cis reservoir during the sequencing of the engaged DNA if interference is a concern. This can be accomplished by a wash step performed before the magnetic field is engaged. The unused DNA can be sequestered in a storage reservoir and may be retained or released by a switchable magnet for later use.
4. The scan plate (510) and nanopore chip (501) are brought relatively close together, with a separation distance less than the flexible linker length. For the λ-DNA flexible linker about 10-15 um is appropriate. The separation is held at this position.
5. The magnet is engaged, and ramped slowly to full force. At some point in the slow ramp-up magnetic force will match and then exceed the nanopore electric force (Fₘ≥Fₑₗ) and the magnetic bead starts to pull the ssDNA out. The ssDNA will continue to be pulled out until the magnetic bead contacts the scan plate directly above the nanopore (i.e. the ssDNA is properly aligned with the nanopore) (FIG. 15c)
6. At this time, some of the ssDNAs may recoil slightly and pull extra bases out of the nanopore. This happens because the magnetic force (Fₘ) will have ramped up somewhat between the time the ssDNA first leaves the nanopore and when it contacts the scan plate. In order to move through the buffer solution the magnetic bead force must match some fluidic drag forces impeding the motion. This will over-stretch the ssDNA slightly. After the recoil, the ssDNA reaches an equilibrium where the tension from its base-to-base stretching that exactly matches the nanopore electric force. (T_{ssDNA}=Fₑₗ)
7. Now, the magnetic force is increased to the maximum to hold the bead tight to the scan plate. The sequencing proceeds by moving the scan plate at the required speed for base recognition. The ssDNA is pre-tensioned and remains under tension during the sequencing process.
8. The remaining length of the λ-DNA (or alternative linker) on the trans side of the nanopore must be sequenced before the sample fragment is read. This is a necessary inefficiency. The λ-DNA could act as the calibration or a custom ssDNA sequence could be used for the linker and sensor calibration. The λ-DNA length that is read can be minimized if the nanopore chip and the scan plate are exactly parallel such that starting separation be only slightly shorter than the stretched flexible linker length.
9. After the sequencing is completed the nanopore bias can be shut off or reversed and the magnet disengaged to allow the tagged DNA to remix with the buffer and repeat the cycle. Again, a secondary magnet or other means can be used to help detach the magnetic beads as described in Method #1, alignment procedure step 5.
   This method has some significant advantages. It is particularly useful that the transition from the linker to the sample DNA is completely smooth. The sample DNA can therefore be analyzed from end to end. If a custom linker molecule is used some calibration sequences could be included that allow each nanopore to be characterized for use in the data analysis. This calibration could be used to record the sensors response to the various transitions from base to base, the response to repeated bases etc. An example of a custom linker molecule is a λ-DNA with calibration sequences added. This additions should be made near the end to which the sample DNA is attached to insure they are recorded.

### Method 3: Alignment Using Direct DNA Attachment

This method has an identical procedure as “Alignment with single stranded DNA Linker” method. The DNA assembly is illustrated in FIG. 13c. The differences are:
There is no long “flexible linker molecule” between the DNA and the magnetic bead. Instead, the sample DNA is attached to the magnetic bead directly:

[magnetic bead]+[ssDNA sample]

A more specific example is:

[1 μm DynaBead® MyOne™ Carboxylic Acid bead]+[amine end modified ssDNA sample]

Another example is:

[1 μm DynaBead® MyOne™ Streptavidin bead]+[Biotinylated ssDNA sample]
Some portion of the DNA cannot be read; Because some portion of the sample DNA must span from the nanopore to the scan plate during the alignment procedure, those bases cannot be properly recorded.
   This approach is simpler and somewhat cheaper than the single-stranded linker method due to the lack of linker molecule. For sequencing of long DNA fragments with a sufficiently large sample of DNA, the inability to sequence the fragments to completion may not present a significant problem. For shorter length DNA fragments or samples with limit material this may severely impede the process.

### Discussion

The automatic alignment method of this disclosure applies to scan plates without micro-pillars (FIGS. 14 & 15) and scan plates with flat bottomed micro-pillars with common or individual cis wells (FIG. 16). The flat end is required such that the magnetic field can pull the magnetic bead (620) directly upward against the micro-pillar without causing a lateral bead motion against the surface.

This magnetic bead assisted DNA attachment to the scan plate makes in-device DNA detaching and reattaching much easier without complicated attaching and detaching chemistry or reagents. The magnetic automated DNA-nanopore alignment makes complicated high precision instrument alignment unnecessary, greatly simplifying the instrument design. Also, the perfect alignment allows all DNA molecules move through the pores at a constant and predictable bases-per-second rate, greatly simplifying the base sensing algorithms.

A requirement of the magnetic alignment method is that the force the magnet applies to the magnetic bead in the scan direction is greater than the electric force acting on the DNA bases inside the nanopore. This may require a large electro-magnet, a large and movable permanent magnet or even an array of permanent magnets. This requirement may dictate the minimum size for the magnetic beads.

Magnetic alignment could be combined with chemical attachment and detachment of the beads and the scan plate. This could be used to minimize the size of the magnetic beads which allows them to remain in suspension longer. Such a procedure could require the nanopore bias voltage to be reduced after the DNA is engaged such that the DNA can be pulled out of the nanopore with the reduced force of a smaller bead. After chemical attachment to the scan plate the nanopore bias voltage could be increased to add to the tension. After the sequencing is completed the DNA could be detached or destroyed.

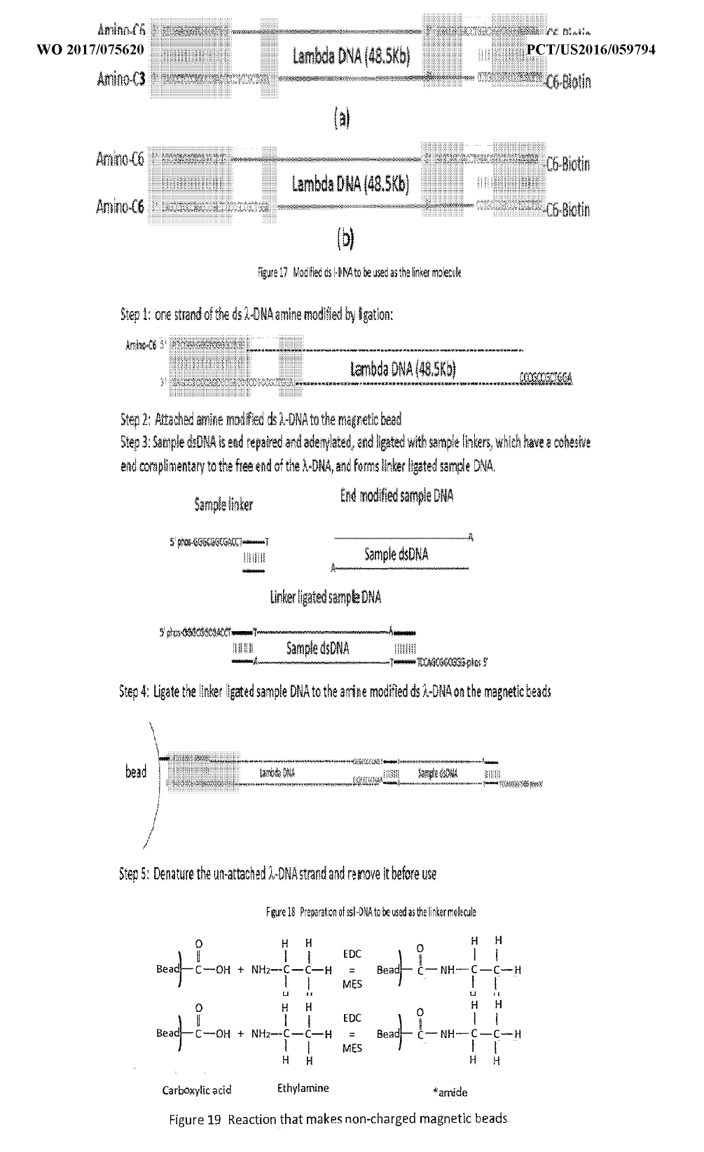

Another means that allows the use of smaller magnetic beads or less strong magnet is to make the magnetic beads un-charged or weakly charged so to minimize non-essential electric force acting on the magnetic beads, especially when the magnetic beads get engaged with the nanopore (FIGS. 13b & 13c and FIG. 15). One solution is to use NeutrAvidin (or other neutral complex) coupled magnetic beads. Since the isoelectric point of NeutrAvidin is 6.3, it behaves almost neutral under normal buffer pH. Another solution is to use the carboxylic acid functionalized magnetic bead. After the reaction of coupling amine-modified DNA or linker molecule, quench the reaction with excessive amount of ethylamine instead of Tris-HCl buffer or ethanolamine, a common suggestion from bead manufacturers. The resulted beads are non-charged but yet hydrophilic. See FIG. 19.

It should be noted in the FIGS. 14 & 15 that the adjustable magnet (520) and the scan plate (510) are shown moving together, this is not an essential feature. The scan plate can be moved independently or the nanopore chip can be moved to alter the separation. The adjustable magnet can be moved independently if necessary.

### Part III: Preventing Cross-Link of DNA, Linker Molecules and Beads

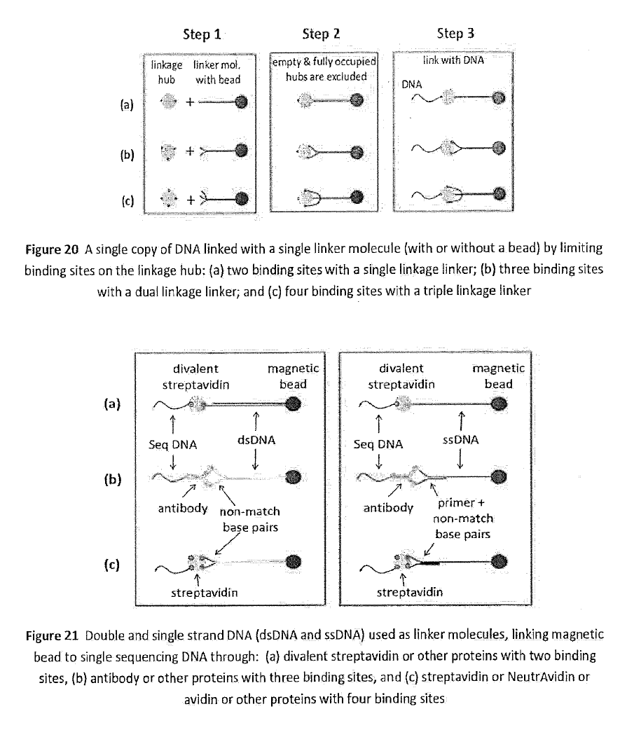

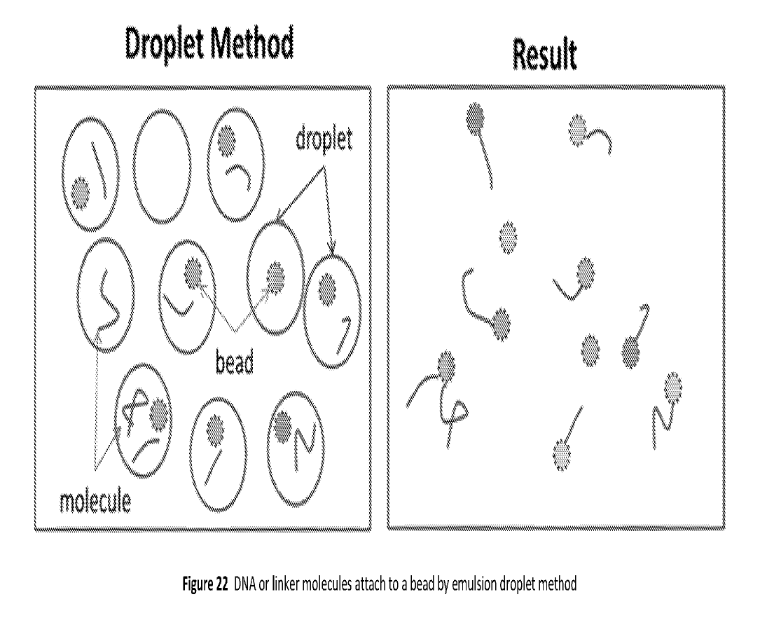

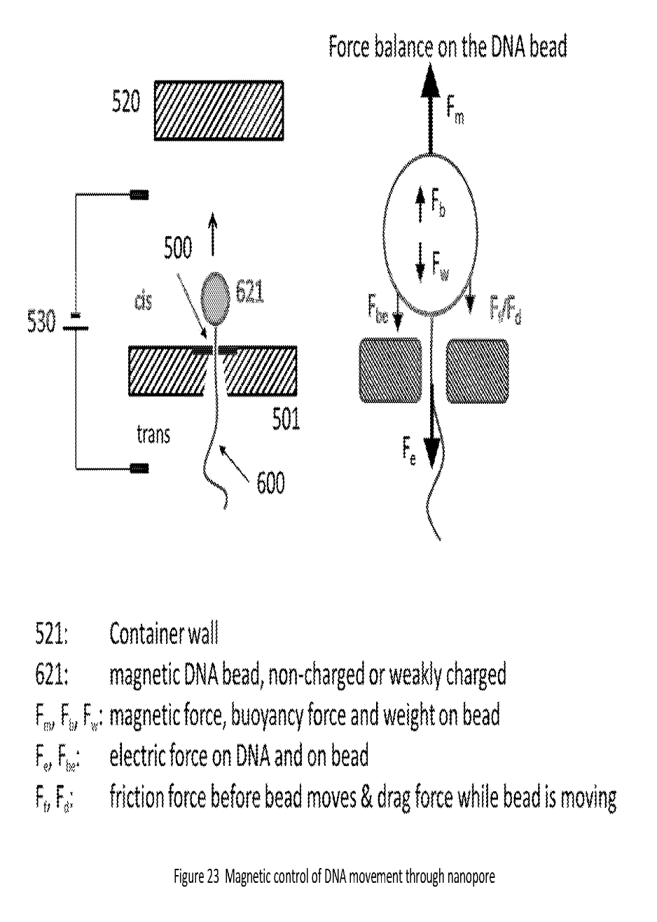

For the above described magnetic bead assisted DNA-nanopore alignment and sequencing approach, a single magnetic bead attached by a single linker molecule attached by a single sample DNA is an embodiment. However, in reality it is difficult to achieve due to the plurality of binding sites on the magnetic bead as well as the linker node. If a linker node of multiple binding sites is used, especially if the linker node is a bead, complicated bead/DNA cross-link network may form, which need be avoided by all means. The following are methods suggested to minimize the creation of complex linker networks:
(1) Control DNA/bead ratio in the mixture:
   Based on the Poisson distribution law, if DNA molecules/fragments are mixed with beads in a 1:1 ratio, it will end up with approximately 36.8% beads having no DNA and among the remaining 63.2% beads with DNA, 87.3% will have 1 or 2 copies and 97% have 1, 2 or 3 copies. If the ratio is reduced to 1:2, we will have approximately only 39.3% bead with DNA but 96.3% of them will have 1 or 2 copies and 99.6% will have 1, 2 or 3 copies. There will be essentially no beads having 4 or more DNA copies (<0.5%). The beads without DNA (null beads) can be removed through enrichment (by free solution electrophoresis or other methods).
(2) Use a linkage protein instead of a linker bead:
   Some large proteins, such as NeutrAvidin and antibodies, are large enough to be used in place of the linker-node bead as the DNA brake at the nanopore. These proteins have very limited binding sites, for example, Neutravidin has only four biotin binding sites and antibody has three branches for binding. Streptavidin and avidin are the same as Neutravidin. There is a divalent streptavidin protein that has only two binding sites, either both next to each other (cis-divalent) or on the opposite side (trans-divalent). For proteins with only two binding sites, we can attach one single-binding linker molecule first and leave only one site for sequencing DNA binding, which guarantees single DNA attachment, see FIG. 20. For proteins with three binding sites, we can attach one dual-binding linker molecule first and again leave only one binding site for the sequencing DNA. And for proteins with four binding sites, such as streptavidin, we can either attach one triple-binding linker molecule first, which leaves one empty binding site, or attach one dual-binding linker molecule and leave two empty binding sites. The chance of getting two sequencing DNA molecules is very small, and also, two copies of DNA link to one magnetic bead (through the linker molecule) is better than one DNA links to two magnetic beads for the automatic alignment scheme. FIG. 21 shows that single strand DNA (ssDNA) and double strand DNA (dsDNA) are used as the linker molecule which provide either a single binding site (ssDNA or dsDNA with only one strand biotinylated) or dual binding sites (dsDNA or ssDNA with hybridized primer at the end). Together with a divalent streptavidin or a regular streptavidin or an antibody, it is able to link only a single sequencing DNA molecule to a magnetic bead.
(3) Emulsion droplet method: use either microfluidic techniques or bulk-shaking or other mixing techniques to generate water-in-oil droplets with size larger than and comparable to the bead size such that one droplet can contain only one bead or at most two beads, together with randomly distributed DNA molecules. At the end the reaction is quenched and the beads with DNA are separated from the free DNA and the empty beads. The result will be similar to method (1) due to the same Poisson distribution law, see FIG. 22, but the cross-link between beads can be effectively avoided.
(4) Directly connect an ssDNA flexible linker molecule to single strand sample DNA:
   As shown in FIGS. 13b & 15, the sample ssDNA is ligated to the ssDNA linker, forming a new continuous single molecule. This method inherently avoids the problem of multiple connections since ssDNA can only connect directly to the end of another ssDNA. There is no complex bead/DNA networking problem with this approach.

### Part IV: Magnetic Control of DNA Movement

This disclosure also provides a method for controlling DNA, RNA and other biological molecules passing through nanopores with a controllable magnet as illustrated in FIGS. 23 & 24. Controlled scan plate movement is not required, which greatly simplifies instrument design and reduces instrument cost. Here, the scan plate is replaced by a container wall (521) placed the same way as the scan plate but fixed at a distance equal to or greater than the maximum dynamic gap between the scan plate and the nanopore substrate as they move.

FIG. 23a shows that the target DNA (600) is attached to a magnetic bead (621) directly. The DNA is driven into a nanopore (500) on the nanopore chip (501) by a bias voltage (530) and stopped by the magnetic bead. An adjustable magnet (520), either an electromagnet or permanent magnet, is turned on or repositioned, starting with near zero magnetic field strength. There are numerous forces acting on the magnetic bead (621) as illustrated in FIG. 23b:
the magnetic force (Fₘ)
the buoyance force (F_{b}) due to displacement of buffer volume
the electrical force acting on the charged DNA (Fₑ)
the electrical force directly acting on the bead due to bead surface charges (F_{be})
the weight of the magnetic bead with DNA attached (F_{w})
the friction force (F_{f}) (exists between the bead and the pore entrance before it moves) or drag force (F_{d}) (exerted on the bead by surrounding buffer when it moves).

Assuming the scan plate and nanopore chip, which are parallel to each other, are in a horizontal position and the bead moves vertically from the nanopore toward the scan plate, the pulling force is the sum of Fₘ & F_{b}, and the opposing force is the sum of Fₑ, F_{be}, F_{w}, F_{f}, and F_{d}. By gradually increasing the magnetic force, the pulling force will eventually surpass the opposing force, see below, so that the DNA can be pulled out of the nanopore.

Fₘ+F_{b}>=Fₑ+F_{be}+F_{w}+F_{f}+F_{d}

For successful base sequencing, the movement of the DNA needs to be slow and controllable. That means the resulting force balance should be near equilibrium and stable. Among all the forces, F_{w} and F_{b} are constant; Fₑ is dependent on the bias voltage, buffer conditions, nanopore conditions and number of DNA bases inside the nanopore and is usually constant; F_{d} is a passive force that exists only when the bead is moving, which stabilizes and dampens out DNA jittering movement; Fₘ is determined by the magnitude of the controllable magnetic field.

The remaining two forces, F_{be} and F_{f}, are location specific and therefore are problematic for achieving the goal of slow and controllable DNA movement. The electric field is very strong near and at the pore but very weak further away from the pore, F_{be} may be strong before the DNA moves but drop to near zero when the bead moves away from the pore. Also, the friction F_{f}, if any, is non-zero at the pore but drops to zero immediately when the bead leaves the pore. As the magnetic bead leaves the immediate vicinity of the nanopore, the sudden imbalance of the forces will cause a sudden accelerated DNA movement.

In order to move the DNA at a slow and smooth predictable speed, the sticking forces (F_{be}+F_{f}) must be completely eliminated or greatly minimized. This can be done by choosing a non-sticking magnetic bead and making the bead non-charged. By reducing the sticking force to zero or nearly zero, the force balance can be precisely controlled, so can the DNA movement.

There are several ways to make a bead non-charged. One is to use NeutrAvidin (or other neutral complex) coupled magnetic beads. Since the isoelectric point of NeutrAvidin is 6.3, it behaves almost neutral under normal buffer pH. Another solution is to use the carboxylic acid functionalized magnetic bead. After the reaction of coupling amine-modified DNA, quench the reaction with excessive amount of ethylamine instead of Tris-HCl buffer or ethanolamine, a common suggestion from bead manufacturers. The resulted beads are non-charged but yet hydrophilic. See FIG. 19.

There will always be a slight imbalance of the forces that causes acceleration of the DNA. This is especially true for a large quantity of beads with a plurality of pores on a nanopore array where the magnetic force Fₘ may vary across the array. The key to keep the DNA movement slow and predictable is to estimate the speed of the DNA from analysis of the base sensing signals and adjust the magnitude of the magnetic field or the bias voltage accordingly. Hence, DNA sequencing can be done with a proper base sensing method and a well-designed computer program and calibration mechanism.

Brownian motion may cause instability of DNA movement. To solve this problem, one option is to lower the buffer temperature and increase buffer viscosity to reduce the magnitude of the Brownian motion. Another option is to increase the electric force Fₑ by using higher bias voltage as long as the electric force is kept below the maximum magnetic force that can be achieved. As the bead moves, the target DNA molecule will be stretched under force, and an intra-molecular tension will be built up, thus the target molecule does not move freely through the nanopore but rather is held taut under the tension from the opposing forces as it progresses, similar to the mechanical control approach.

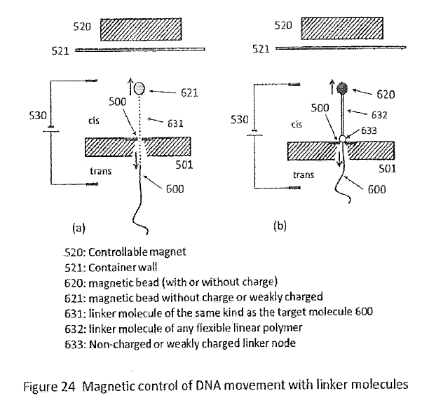

Furthermore, a linker molecule of the same kind of the target molecule can be used to link the target molecule to the bead. For example, a single strand λ-DNA can be used as the linker and ligated to the sample ssDNA, which forms a long continuous ssDNA, as shown in FIG. 13b and FIG. 24a. The linker molecule may be served as a calibration tool, and in the same time it can provide a buffer zone so that any rapid acceleration of DNA movement at the start can be dampened out before the actual DNA sample is sequenced.

As an alternative, a linker node (633) is introduced as the connection between the linker molecule (632) and the target molecules (600) (FIG. 24b). The linker node is at least twice as large as the nanopore entrance so that it will function as the stopper or brake as the DNA translocates through the nanopore under the bias voltage. Since now the linker node is at the nanopore, it is required to be non-charged in the sequencing buffer while the magnetic bead (620) is not required to be non-charged. The linker node can be a large protein (such as an antibody, NeutrAvidin, or streptavidin, etc.), or any polymer complex larger than the nanopore entrance or even a non-magnetic bead.

### REFERENCES

1. Akeson M A, Deamer D W and Branton D, Miniature support for thin films containing single channels or nanopores and methods for using same, U.S. Pat. No. 6,267,872, 2001

2. Akeson M, Branton D, Deamer D W, Sampson J R, Methods and apparatus for characterizing polynucleotides, U.S. Pat. No. 7,947,454, 2011

3. Bayley H, Astier Y, Braha O, “Methods using pores”, U.S. Pat. No. 8,105,846, 2012

4. Derrington I M, Butler T Z, Collins M D, Manrao E, Pavlenok M, Niederweis M and Gundlach J H, Nanopore DNA sequencing with MspA, PNAS 2010, 107 (37):16060-16065.

5. Fixe F, Dufva M, Telleman P and Christensen C B V, Functionalization of poly(methyl methacrylate) (PMMA) as a substrate for DNA microarrays, Nucleic Acids Research, 2004, 32 (1):e9 (DOI: 10.1093/nar/gng157)

6. Fologea D, Uplinger J, Thomas B, McNabb D S and Li J, Slowing DNA translocation in a solid state nanopore, Nano Lett., 2005, 5 (9): 1734-1737.

7. Goyal P, Krasteva P V, Gerven N V, Gubellini F, Broeck I V D, Troupiotis-Tsailaki A, Jonckheere W, Pehau-Arnaude G, Pinkner J S, Chapman M R, Hultgren S J, Howorka S, Fronzes R and Remaut H, Structural and mechanistic insights into the bacterial amyloid secretion channel CsgG, Nature 2014, 516 (7530):250-253.

8. Hall A R, Scott A, Rotem D, Mehta K K, Bayley Hagan and Dekker C, Hybrid pore formation by directed insertion of a-haemolysin into solid-state nanopores, Nature Nanotechnology 2010, 5:874-877.

9. Han S J, Royyuru A K, Stolovitzky G and Wang D, Integrated nanowire/nanosheet nanogap and nanopore for DNA and RNA sequencing, Patent# US2014/0326954 A1, 2014.

10. Holmberg A, Blomstergren A, Nord O, Lukacs M, Lundeberg J and Uhlen M, The biotin-streptavidin interaction can be reversibly broken using water at elevated temperatures, Electrophoresis 2005, 26 (3):501-510.

11. Kasianowicz J J, Brandin E, Branton D and Deamer D W, Characterization of individual polynucleotide molecules using a membrane channel, Proc. Natl. Acad. Sci. USA, 1996, 93, 13770-13773. Keyser U F, Controlling molecular transport through nanopores, J. R. Soc. Interface 2011, 8:1369-1378.

12. Keyser U F, van der Does J, Dekker C and Dekker N H, Optical tweezers for force measurements on DNA in nanopores. Nat. Phys, 2006, 2:473-477.

13. King G M and Golovchenko J A, Probing nanotube-nanopore interactions, Phy Rev Let 2005, 95, 216103

14. Kowalczyk S W, Wells D B, Aksimentiev A, and Dekker C, Slowing down DNA translocation through a nanopore in Lithium Chloride, Nano Letters 2012, 12 (2), pp 1038-1044

15. Lindsay S and Zhang P M, Devices and methods for target molecule characterization, Patent #: WO 2008/124707 A9, 2008.

16. Lindsay S and Zhang P M, Devices and methods for target molecule characterization, U.S. Pat. No. 9,395,352 B2, 2016

17. Liu Y and Rauch C B, DNA probe attachment on plastic surfaces and microfluidic hybridization array channel devices with sample oscillation, Analytical Biochemistry 2003, 317:76-84.

18. Lu B, Albertorio F, Hoogerheide D P and Golovchenko J A, Origins and consequences of velocity fluctuations during DNA passage through a nanopore, Biophysical J. 2011, 101:70-79.

19. Lu B, Fleming S, Szalay T and Golovchenko J A, Thermal motion of DNA in an MspA pore, BioRxiv 2015 (dx.doi.org/10.1101/021766)

20. Peng H and Ling X S, Reverse DNA translocation through a solid-state nanopore by magnetic tweezers, Nanotechnology, 2009, vol 20, 185101.

21. Peng H, Stolovitsky G A, Rossnagel S M, Polonsky S, Luan B, Martyna G J, Piezoelectric-based nanopore device for the active control of the motion of polymers through the same, U.S. Pat. No. 0,223,652, 2011

22. Polonsky S, Rossnagel S and Stolovitzky G, Nanopore in metal-dielectric sandwich for DNA position control, Applied Physics Letters 2007, vol 91, no 153103

23. Polonsky S, Rossnagel S M, Stolovitzky G A, Systems and methods for controlling position of charged polymer inside nanopore, U.S. Pat. No. 8,003,319, 2011

24. Sassolas A, Leca-Bouvier B D and Blum U, DNA Biosensors and Microarrays, Chem. Rev. 2008, 108:109-139.

25. Sauer-Budge A F, Nyamwanda J A, Lubensky D K, and Branton D, Unzipping kinetics of double-stranded DNA in a nanopore. Phys Rev Lett 2003, 90:238101.

26. Singh V, Zharnikov M, Gulino A, Gupta T, DNA immobilization, delivery and cleavage on solid supports, J. Mater. Chem., 2011 21:10602-10618.

27. Stoddart D, Franceshini L, Heron A, Bayley H and Maglia G, DNA stretching and optimization of nucleobase recognition for enzymatic nanopore sequencing, Nanotechnology, 2015, 26 (8):084002

28. Tsutsui M, He Y, Furuhashi M, Rahong S, Taniguchi M and Kawai T, Transverse electric field dragging of DNA in a nanochannel, Scientific Reports, 2012, 2:394-

[symbol]

29. Wanunu M, Nanopores: a journey towards DNA sequencing, Physics of Life Reviews, 2012, 9:125-158.

30. Wanunu M and Meller A, Chemically modified solid-state nanopores, Nano Lett, 2007, 7:1580-1585.

31. Yeh L H, Zhang M, Joo S W and Qian S, Slowing down DNA translocation through a nanopore by lowering fluid temperature, Electrophoresis, 2012, 33 (23):3458-65.

1. A system for controlling movement of a charged linear molecule comprising:
a substrate positioned between a cis space and a trans space;
a nanopore in the substrate through which at least a portion of the charged linear molecule can pass from the cis space to the trans space;
a scan plate located in the cis space to which directly or indirectly a first end of the charged linear molecule is attached;
an actuator for controlling the distance between the substrate and the scan plate such that they can be moved with nanometer precision; and
a bias source for applying a bias voltage between the cis space and the trans space to direct a second end of the charged linear molecule to enter into the nanopore.

2. The system of claim 1, further comprising
an attachment system that assists the attachment of the charged linear molecule to the scan plate such that the charged linear molecule can move with the scan plate.

3. The system of claim 1,
wherein the substrate is a nanopore chip comprising a plurality of nanopores positioned in a planar arrangement wherein each nanopore is substantially equidistant from the surface of the scan plate.

4. The system of claim 1,
wherein the nanopore comprises a biological pore, or a synthetic pore, or a combination thereof.

5. The system of claim 4,
wherein the biological pore is selected from the group consisting of an alpha-hemolysin pore, a MspA pore, a CsgG pore, a modified version thereof, and a combination thereof.

6. The system of claim 4,
wherein the synthetic pore is made from silicon nitride (Si₃N₄), silicon dioxide (SiO₂), aluminum oxide (Al₂O₃), boron nitride (BN), graphene, molybdenum disulfide (MoS2) or a polymer or a hybrid thereof.

7. The system of claim 2,
wherein the attachment system comprises a chemical bond, either covalent or non-covalent, and either reversible or non-reversible.

8. The system of claim 7,
wherein the chemical bond is selected from the list comprising a biotin-streptavidin bond, an amide bond; a phosphodiester bond, ester bond, disulfide bond, imine bond, aldehyde bond, hydrogen bond, hydrophobic bonds, and a combination thereof.

9. The system of claim 2,
wherein the attachment system comprises a magnetic bead, which is attached to the first end of the charged linear molecule; and
wherein the magnetic bead is made from one of the following materials: (a) paramagnetic, (b) super-paramagnetic, (c) ferromagnetic, or (d) diamagnetic.

10. The system of claim 9, further comprising
a controllable magnet comprising an electromagnet, an adjustable permanent magnet, a group of magnets, or a combination thereof;
wherein the controllable magnet is configured to attract the magnetic bead towards the scan plate and to hold the magnet bead against the scan plate.

11. The system of claim 2,
wherein the attachment system comprises a flexible linker molecule, and the flexible linker molecule is attached to the first end of the charged linear molecule at one end and attached to the scan plate at other end.

12. The system of claim 11,
wherein the flexible linker molecule is selected from the group consisting of a single stranded nucleic acid, a double stranded nucleic acid, a polypeptide chain, a cellulose fiber or any flexible linear polymer, either natural, modified or synthesized, and a combination thereof.

13. The system of claim 11,
wherein the flexible linker molecule is the same kind of molecule as the charged linear molecule.

14. The system of claim 11,
wherein the attachment system further comprises a linker node which is disposed between the flexible linker molecule and the charged linear molecule; and
wherein the linker node is configured to block the linker molecule from entering the nanopore.

15. The system of claim 14,
wherein the linker node is a protein selected from the group consisting of an antibody, an enzyme, a NeutrAvidin, a streptavidin, and an avidin, or a polymer complex, or a particle or a bead, or a combination thereof.

16. The system of claim 9,
wherein a flexible linker molecule is disposed between the charged linear molecule and the magnetic bead.

17. The system of claim 16,
wherein the flexible linker molecule is selected from the group consisting of a single stranded nucleic acid, a double stranded nucleic acid, a polypeptide chain, a cellulose fiber or any flexible linear polymer, either natural, modified or synthesized, and a combination thereof.

18. The system of claim 16,
wherein the flexible linker molecule is the same kind of molecule as the charged linear molecule.

19. The system of claim 16,
wherein the attachment system further comprises a linker node which is disposed between the flexible linker molecule and the charged linear molecule; and
wherein the linker node is configured to block the linker molecule from entering the nanopore.

20. The system of claim 19,
wherein the linker node is a protein selected from the group consisting of an antibody, an enzyme, a NeutrAvidin, a streptavidin, an avidin, a polymer complex, a particle, a non-magnetic bead, and a combination thereof.

21. The system of claim 1 further comprising
a detector for determining the identity or characteristics of individual base units of the charged linear molecule as they pass through the nanopore, wherein the base units of the charged linear molecule can be detected by their effect on the ionic current blockage, or recognition tunneling, or field-effect transistor, or other base sensing methods, or a combination thereof.

22. The system of claim 1 further comprising
a micro-pillar attached or microfabricated onto the scan plate that has either a pointed end or a flat-bottom end, which is configured to allow the attachment of the first end of the charged linear molecule.

23. The system of claim 20,
wherein the micro-pillar is an array of micro-pillars on the scan plate, laterally positioned to match an array of nanopores on the substrate.

24. The system of claim 1
wherein the actuator comprises a precision linear motion stage that is configured to control the distance between the scan plate and the substrate such that the charged linear molecule can be pulled out or inserted into the nanopore at a steady rate that enables accurate base unit sequencing.

25. The system of claim 24,
wherein the rate is about 0.5 ms per base unit or slower.

26. The system of claim 25,
wherein the rate is from about 3 ms to about 20 ms per base unit.

27. The system of claim 24,
wherein the precision linear motion stage comprises a linear stage driven by a piezo-electric effect drive with nanometer or sub-nanometer precision.

28. The system of claim 1,
wherein the actuator comprises a coarse precision actuator that is coupled to the scan plate or the substrate by mechanical reduction allowing for nanometer or sub-nanometer precision movement of the scan plate or the substrate.

29. The system of claim 28,
wherein the coarse precision actuator comprises a micrometer or a sub-micrometer servo motor

30. The system of claim 1, further comprising
an adjustment stage with micrometer precision that is coupled to the scan plate or the substrate that is configured to move the object laterally and/or vertically for pre-sequencing position adjustment.

31. The system of claim 1,
wherein a plurality of charged linear molecules are attached to the scan plate randomly.

32. The system of claim 1
wherein a plurality of charged linear molecules are attached to a patterned area on the scan plate, wherein the plurality of charged linear molecules in the patterned area is laterally aligned with a plurality of nanopores on the substrate.

33. The system of claim 10, further comprising
a secondary adjustable magnet that is configured to remove the magnetic bead from the scan plate.

34. The system of claim 1
wherein the charged linear molecule is a nucleic acid sequence or a polypeptide sequence; and
wherein the nucleic acid sequence is selected from the list consisting of single stranded DNA, double stranded DNA, single stranded RNA, oligonucleotide, a sequence comprising a modified nucleotide, and a combination thereof.

35. A method for controlling movement of a charged linear molecule comprising
providing a scan plate and a substrate plate placed substantially parallel and aligned laterally with each other;
attaching a first end of the charged linear molecule to the scan plate, either directly or indirectly;
aligning the charged linear molecule with a nanopore in the substrate plate by an adjustable mechanical apparatus;
directing a second end of the charged linear molecule to the nanopore in the substrate plate by an electric force;
moving the charged linear molecule through the nanopore by adjusting the distance between the scan plate and the substrate plate;
maintaining an intra-molecular tension in the charged linear molecule by adjusting the electric force during the sequencing process.

36. The method of claim 35,
wherein the adjustable mechanical apparatus comprises a single axis or a multi-axis linear stage with micrometer or sub micrometer precision.

37. The method of claim 35,
wherein adjusting the distance between the scan plate and the substrate plate comprises moving the scan plate and/or the substrate plate with an actuator.

38. The method of claim 37,
wherein the actuator comprises a linear stage driven by a piezo-electric effect drive with nanometer or sub-nanometer precision.

39. The method of claim 37,
wherein the actuator comprises a coarse precision actuator that is coupled to the scan plate or the substrate by mechanical reduction providing for nanometer or sub-nanometer precision movement of the scan plate or the substrate plate.

40. The method of claim 39,
wherein the coarse precision actuator comprises a micrometer or a sub-micrometer precision servo meter.

41. The method of claim 35,
wherein the electrical force is achieved by an electrical bias apparatus which is configured to apply a bias voltage across the substrate plate allowing a current through the nanopore and is further configured to pull the charged linear molecule through the nanopore.

42. The method of claim 35,
wherein a flexible linker molecule is disposed between the charged linear molecule and the scan plate; and
wherein the flexible linker molecule is selected from the list consisting of a single stranded nucleic acid, a double stranded nucleic acid, a polypeptide chain, a cellulose fiber or any flexible linear polymer, either natural, modified or synthesized, and a combination thereof.

43. The method of 42,
wherein a linker node is disposed between the linker molecule and the charged linear molecule and the linker node is configured to block the linker molecule from entering the nanopore, and
wherein the linker node is a protein selected from the list consisting of an antibody, an enzyme, a NeutrAvidin, a streptavidin, and an avidin, or a polymer complex or particle or bead, a portion thereof, and a combination thereof.

44. The method of claim 35, further comprising
attaching the first end of the charged linear molecule to a magnetic bead;
wherein the magnetic bead is chosen from the list consisting of a super-paramagnetic bead, a paramagnetic bead, a ferromagnetic bead, and a diamagnetic bead;
aligning the charged linear molecule with the nanopore by applying a magnetic field to attract the magnetic bead towards the scan plate while the electric force maintains the engagement of the charged linear molecule with the nanopore;
wherein the magnetic field is from an electromagnet or an adjustable permanent magnet, or a group of magnets, or a combination thereof;
wherein the magnetic bead contacts the surface of the scan plate substantially orthogonally aligned above the nanopore and is held tightly against the scan plate by the magnetic field such that the magnetic bead and the charged linear molecule move substantially with the scan plate.

45. The method of claim 44,
wherein a flexible linker molecule is disposed between the charged linear molecule and the magnetic bead; and
wherein the flexible linker molecule is selected from the list consisting of a single stranded nucleic acid, a double stranded nucleic acid, a polypeptide chain, a cellulose fiber or any flexible linear polymer, and a combination thereof, either natural, modified or synthesized.

46. The method of claim 45,
wherein a linker node is disposed between the linker molecule and the charged linear molecule and the linker node is configured to block the linker molecule from entering the nanopore; and
wherein the linker node is a protein selected from the list consisting of an antibody, an enzyme, a NeutrAvidin, a streptavidin, and an avidin, or a polymer complex or a non-magnetic particle/bead, or a combination thereof; and
wherein aligning the charged linear molecule with a nanopore in the substrate plate further comprises
setting a distance between the scan plate and the substrate plate larger than the length of the linker molecule;
applying a magnetic field such that the magnetic force on the magnetic bead is sufficient to lift the magnetic bead, but not sufficient to substantially lift the linker node;
wherein the linker node is engaged at the nanopore by the electric force,
reducing the distance between the scan plate and the substrate plate to be smaller than the length of the linker molecule;
wherein the magnetic bead contacts the scan plate and is held by the scan plate substantially orthogonally aligned above the nanopore, resulting in a substantial orthogonal alignment between the bead, the linker molecule and the charged linear molecule.

47. The method of claim 35,
wherein the charged linear molecule is randomly distributed on the scan plate, either attached directly or indirectly.

48. The method of claim 35,
wherein a plurality of the charged linear molecules is distributed in a patterned area on the scan plate either attached directly or attached indirectly, wherein the plurality of the charged linear molecules in the patterned area on the scan plate is substantially laterally aligned with a plurality of the nanopores on the substrate plate.

49. The method of claim 35,
wherein the substrate plate is a nanopore chip comprising a plurality of nanopores positioned in a planar arrangement such that each nanopore is substantially equidistant from the surface of the scan plate.

50. The method of claim 35,
wherein the scan plate has a micro-pillar facing the nanopore, which has either a pointed end or a flat end; and
wherein the first end of the charged linear molecule is attached to the micro-pillar, either directly or indirectly.

51. The method of claim 50,
wherein the scan plate has an array of micro-pillars that match an array of nanopores on the substrate plate.

52. The method of claim 35,
wherein the charged linear molecule is a nucleic acid sequence or a polypeptide sequence; and
wherein the nucleic acid sequence is selected from the list consisting of single stranded DNA, double stranded DNA, single stranded RNA, oligonucleotide, a sequence comprising a modified nucleotide, and a combination thereof.
